(19) United States
(12) Patent Application Publication
(10) Pub. No.: US 2022/0112162 A1
(43) Pub. Date: Apr. 14, 2022

(54) ALKYL QUATERNARY AMMONIUM TRYPTAMINES AND THEIR THERAPEUTIC USES

(71) Applicant: CAAMTECH, INC., Issaquah, WA (US)

(72) Inventor: Andrew R. CHADEAYNE, Issaquah, WA (US)

(73) Assignee: CAAMTECH, INC., Issaquah, WA (US)

(21) Appl. No.: 17/495,069

(22) Filed: Oct. 6, 2021

Related U.S. Application Data
(63) Continuation-in-part of application No. PCT/US2020/047791, filed on Aug. 25, 2020.
(60) Provisional application No. 62/891,388, filed on Aug. 25, 2019.
(60) Provisional application No. 63/007,653, filed on Apr. 9, 2020.
(60) Provisional application No. 63/053,976, filed on Jul. 20, 2020.
(60) Provisional application No. 63/139,976, filed on Jan. 21, 2021.

(51) Int. Cl. C07D 209/16; A61K 45/06

(52) U.S. Cl. CPC C07D 209/16; A61K 45/06

(57) ABSTRACT

The disclosure relates to a compound of formula (I):

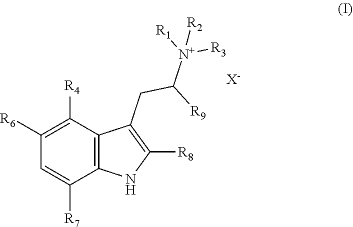

This disclosure also relates to a compound for formula (II):

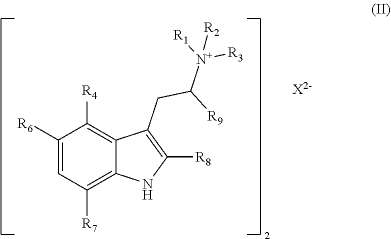

This disclosure also relates to a compound of formula (III):

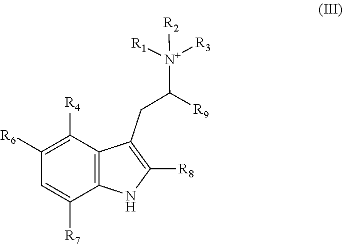

The disclosure also relates to crystalline compounds of formula (I), (II), or (III). The disclosure relates to compositions comprising, consisting essentially of, or consisting of a compound of formula (I), (II), or (III) and an excipient. The disclosure also relates to pharmaceutical compositions comprising a therapeutically effective amount of a compound of formula (I), (II), or (III) where the excipient is a pharmaceutically acceptable carrier. The disclosure further relates to therapeutic uses of compounds of formula (I), (II), or (III).

### RELATED APPLICATIONS

This application is a Continuation-in-Part that claims priority to PCT International Application No. PCT/US2020/047791, filed on Aug. 25, 2020, which claims priority to U.S. Provisional Application No. 62/891,388, filed on Aug. 25, 2019, U.S. Provisional Application No. 63/007,653, filed on Apr. 9, 2020, and U.S. Provisional Application No. 63/053,976, filed on Jul. 20, 2020; and this application claims priority to U.S. Provisional Application No. 63/139,976, filed on Jan. 21, 2021; each of which are incorporated by reference.

### TECHNICAL FIELD

This disclosure relates to alkyl quaternary ammonium tryptamines, compositions and pharmaceutical compositions containing them as well as their use in treating various diseases.

### BACKGROUND

N,N-dimethyltryptamine (DMT) and its derivatives have been used by humans for centuries because of their psychoactive, entheogenic, or hallucinogenic effects, or combinations thereof (Cameron & Olson, 2018). Psilocybin, the 4-phosphate variant of DMT, is arguably its most studied derivative. Psilocybin is one of several naturally occurring psychoactive tryptamines found in “magic” mushrooms. When consumed by humans, psilocybin serves as a prodrug of psilocin. Upon digestion, psilocybin hydrolyses to generate psilocin, the 4-hydroxy derivative of DMT. Psilocin is a potent serotonin 2a-agonist, which is responsible for its psychoactive properties (Dinis-Oliveira, 2017; Nichols, 2012).

Psychoactive tryptamines like DMT and psilocin have garnered significant interest recently because of their potential for treating mood disorders, including depression, anxiety, addiction, and post-traumatic stress disorder (PTSD) (Johnson & Griffiths, 2017; Carhart-Harris & Goodwin, 2017). Altering the chemical structure within this class of compounds can dramatically influence the potency and action of the drugs. For example, merely changing the N,N-dialkyl groups on DMT can modify its psychoactive properties: increasing the chain length of the two alkyl groups of the tryptamine to larger than n-butyl dramatically reduces or eliminates the psychoactive effects (Bradley & Johnston, 1970).

The synthesis of N-methyl-N-isopropyltryptamine (MiPT) was reported in 1981 (Repke et al., 1981). In 1985, Repke and co-workers reported that of the compounds in the series of N,N-dialkyl-4-hydroxytryptamines, the N-methyl-N-isopropyl derivative (4-HO-MiPT) is the most potent based upon qualitative effects on humans (Repke et al., 1985). Later quantitative studies showed the N-methyl-N-isopropyl derivatives of DMT and psilocin to be more potent as serotonin-1A, -2A and -2B receptors compared to the analogous dimethyl compounds (McKenna et al., 1990).

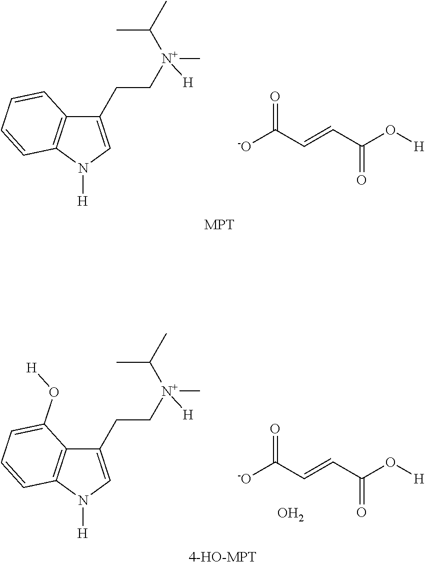

New psychoactive tryptamines have been identified in “magic mushrooms” as recently as 2017. (Lentz, et al., 2017.) Until this year, there was no general synthetic method for producing useful amounts of the minor psychoactive tryptamines. (Sherwood, Halberstadt, et al.) One of these minor components is aeruginascin, (Jensen, et al., 2006) the N-trimethyl analogue of psilocybin. The limited exposure of humans to Inocybe aeruginascens mushrooms, the only known species in which aeruginascin has been found, has resulted in hallucinations that exhibited only euphoric experiences. (Gartz, 1989). This is in contrast to psilocybin and psilocin mushrooms, which often lead to dysphoric moods during the psychedelic experience. Despite these observations, the pharmacological activity of aeruginascin has remained unexplored.

Even with this previous work, there is a need to develop new psilocybin derivatives with improved properties for treatment of psychological disorders.

### SUMMARY OF THE DISCLOSURE

The disclosure relates to a compound of formula (I):

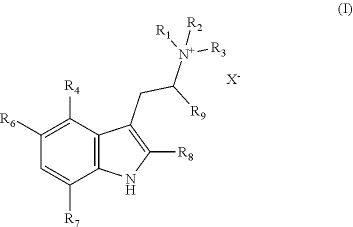

wherein
   R₁, R₂ and R₃ are each independently a straight chain or branched C₁-C₆ alkyl or C₂-C₆ alkenyl;
   R₄ is hydrogen, hydroxyl, C₁-C₆ alkoxy, —OC(O)R₅ or —OC(O)OR₅;
   R₅ is a straight chain or branched C₁-C₆ alkyl;
   R₆ is hydrogen, hydroxyl, C₁-C₆ alkoxy, —OC(O)R₅, —OC(O)OR₅, or a straight chain or branched C₁-C₆ alkyl;
   R₇, R₈ and R₉ are each independently hydrogen or a straight chain or branched C₁-C₆ alkyl; and
   X⁻ is a pharmaceutically acceptable anion.

The disclosure also relates to a compound of formula (II):

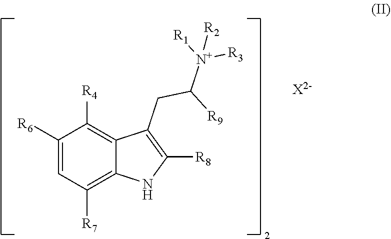

wherein
   R₁, R₂ and R₃ are independently selected from straight chain or branched C₁-C₆ alkyl or a straight chain or branched C₂-C₆ alkenyl;
   R₄ and R₆ are independently chosen from hydrogen, hydroxyl, —OR₅, —OC(O)R₅, and —OC(O)OR₅;
   R₅ is a straight chain or branched C₁-C₆ alkyl or a substituted or unsubstituted aryl;
   R₇, R₈ and R₉ are each independently hydrogen or a straight chain or branched C₁-C₆ alkyl; and
   X²⁻ pharmaceutically-acceptable dianion.

The disclosure also relates to a zwitterionic compound of formula (III):

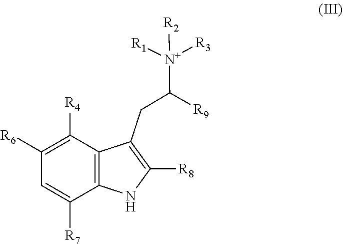

wherein
   R₁, R₂ and R₃ are each independently a straight chain or branched C₁-C₆ alkyl or C₂-C₆ alkenyl;
   R₄ is hydrogen, —O⁻, C₁-C₆ alkoxy, —OC(O)R₅, —OC(O)O⁻, —OSO₂O⁻, or —OP(O)OHO⁻;
   R₅ is a straight chain or branched C₁-C₆ alkyl;
   R₆ is hydrogen, —O⁻, C₁-C₆ alkoxy, —OC(O)R₅, —OC(O)OR₅, or a straight chain or branched C₁-C₆ alkyl; and
   R₇, R₈ and R₉ are each independently hydrogen or a straight chain or branched C₁-C₆ alkyl,
wherein at least one of R₄ or R₆ is —O⁻, —OC(O)O⁻, —OSO₂O⁻, or —OP(O)OHO⁻, provided that R₄ is not —OP(O)OHO⁻ when R₁, R₂ and R₃ are each methyl.

The disclosure relates to compositions comprising, consisting essentially of, or consisting of a compound of formula (I), formula (II), or formula (III), and an excipient. The disclosure also relates pharmaceutical compositions comprising a therapeutically effective amount of a compound of formula (I) where the excipient is a pharmaceutically acceptable carrier. The disclosure further relates to a method of preventing or treating a psychological disorder comprising the step of administering to a subject in need thereof a therapeutically effective amount of a compound of formula (I) or of a pharmaceutical composition containing the compound.

The disclosure also relates to a composition comprising, consisting essentially of, or consisting of as a first active component: a compound of formula (I), formula (II), or formula (III) of the disclosure; and as a second active component selected from (a) a serotonergic drug, (b) a purified psilocybin derivative, (c) one or two purified cannabinoids and (d) a purified terpene, (e) an adrenergic drug, (f) a dopaminergic drug, (g) a monoamine oxidase inhibitor, (h) a purified erinacine, and (i) a purified hericenone; and a pharmaceutically acceptable excipient.

The disclosure also relates to methods of preventing or treating inflammation and/or pain, preventing or treating a neurological disorder, modulating activity of a mitogen activating protein (MAP), modulating neurogenesis, or modulating neurite outgrowth comprising the step of administering to a subject in need thereof a therapeutically effective amount of a compound formula (I), formula (II), or formula (III), and to administering a pharmaceutical composition or a composition according to the disclosure.

The disclosure also relates to a method of generating a dialkyltryptamine compound in situ in a patient, the method comprising administering at least one quaternary tryptamine compound selected from formulas (I), (II) and (III) to the patient. The disclosure also relates to methods of generating a dialkyltryptamine compound comprising contacting at least one quaternary tryptamine compound selected from formulas (I), (II) and (III) with an enzyme in vitro or in vivo, such as an enzyme capable of nitrogen dealkylation (e.g., cytochrome P450 enzymes (CYPs)). The disclosure further relates to methods of generating a dialkyltryptamine compound in situ in a patient comprising contacting at least one quaternary tryptamine compound selected from formulas (I), (II) and (III) with an enzyme in the patient capable of nitrogen dealkylation (e.g., cytochrome P450 enzymes (CYPs)).

### BRIEF DESCRIPTION OF THE FIGURES

FIG. 1 is the fully labeled displacement ellipsoid representation (50%) of the asymmetric unit of crystalline 4-AcO-TMT iodide.

FIG. 2 is the simulated X-ray Powder Diffraction Pattern (XRPD) for 4-AcO-TMT iodide from its single crystal structure.

FIG. 3 is the fully labeled displacement ellipsoid representation (50%) of the asymmetric unit of crystalline 4-FIO-TMT iodide.

FIG. 4 is the simulated X-ray Powder Diffraction Pattern (XRPD) for 4-AcO-TMT iodide from its single crystal structure.

FIG. 5 shows the molecular structure of crystalline DMPT iodide.

FIG. 6 shows the packing of crystalline DMPT iodide.

FIG. 7 is a simulated x-ray powder diffraction (XRPD) pattern of crystalline DMPT iodide from its single crystal data.

FIG. 8 shows the molecular structure of crystalline DMALT iodide.

FIG. 9 shows the packing of crystalline DMALT iodide.

FIG. 10 is a simulated x-ray powder diffraction (XRPD) pattern of crystalline DMALT iodide from its single crystal data.

FIG. 11 shows the molecular structure of crystalline 4-AcO-DMET iodide hemihydrate showing the atomic labeling.

FIG. 12 is a simulated x-ray powder diffraction (XRPD) pattern of crystalline 4-AcO-DMET iodide hemihydrate from its single crystal data.

FIG. 13 shows the molecular structure of crystalline 4-AcO-DMPT iodide showing the atomic labeling.

FIG. 14 is a simulated x-ray powder diffraction (XRPD) pattern of crystalline 4-AcO-DMPT iodide from its single crystal data.

FIG. 15 shows the molecular structure of 4-FIO-DMPT iodide showing the atomic labeling.

FIG. 16 is a simulated x-ray powder diffraction (XRPD) pattern of crystalline 4-FIO-DMPT iodide from its single crystal data.

### DETAILED DESCRIPTION

Compounds of the Disclosure

This disclosure relates to alkyl quaternary tryptammonium compounds of formula (I):

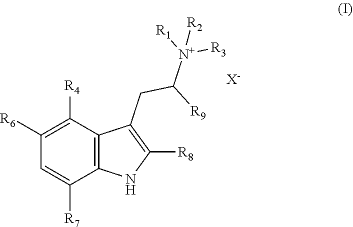

wherein
   R₁, R₂ and R₃ are each independently a straight chain or branched C₁-C₆ alkyl or C₂-C₆ alkenyl;
   R₄ is hydrogen, hydroxyl, C₁-C₆ alkoxy, —OC(O)R₅ or —OC(O)OR₅;
   R₅ is a straight chain or branched C₁-C₆ alkyl;
   R₆ is hydrogen, hydroxyl, C₁-C₆ alkoxy, —OC(O)R₅, —OC(O)OR₅, or a straight chain or branched C₁-C₆ alkyl;
   R₇, R₈ and R₉ are each independently hydrogen or a straight chain or branched C₁-C₆ alkyl; and
   X⁻ is a pharmaceutically acceptable anion.

In formula (I) R₁, R₂ and R₃ are each independently a straight chain or branched C₁-C₆ alkyl, for example a straight chain C₁-C₆ alkyl, or a C₂-C₆ alkenyl, for example allyl. In some embodiments, R₁, R₂ and R₃ are each independently a straight chain or branched C₁-C₄ alkyl, for example a straight chain C₁-C₄ alkyl, or a C₂-C₄ alkenyl. R₁, R₂ and R₃ may each independently be selected from methyl, ethyl, n-propyl, isopropyl, n-butyl, isobutyl and tert-butyl. In other embodiments, R₁, R₂ and R₃ are each methyl, are each ethyl, or a mixture of methyl and ethyl groups.

In formula (I) R₄ is hydrogen, hydroxyl, C₁-C₆ alkoxy, —OC(O)R₅ or —OC(O)OR₅. When R₄ is a C₁-C₆ alkoxy group, or in some embodiments a C₁-C₄ alkoxy group, it may be a straight chain or branched C₁-C₆ alkoxy group or C₁-C₄ alkoxy group, for example a straight chain, and may be methoxy or ethoxy. R₅ is a straight chain or branched C₁-C₆ alkyl or C₁-C₄ alkyl, for example a straight chain C₁-C₄ alkyl. In some embodiments, R₅ is selected from methyl, ethyl, n-propyl or n-butyl, and for example is methyl or ethyl.

In formula (I) R₆ is hydrogen, hydroxyl, C₁-C₆ alkoxy, —OC(O)R₅, —OC(O)OR₅, or a straight chain or branched C₁-C₄ alkyl. When R₄ is a C₁-C₆ alkoxy group, or in some embodiments a C₁-C₄ alkoxy group, it may be a straight chain or branched C₁-C₆ alkoxy group or C₁-C₄ alkoxy group, for example a straight chain, and may be methoxy or ethoxy. R₅ is a straight chain or branched C₁-C₆ alkyl or C₁-C₄ alkyl, for example a straight chain C₁-C₄ alkyl. In some embodiments, R₅ is selected from methyl, ethyl, n-propyl or n-butyl, and for example is methyl or ethyl. When R₆ is a C₁-C₄ alkyl group, it may be methyl, ethyl, propyl, butyl, vinyl, isopropyl, tert-butyl, sec-butyl, or isobutyl.

In formula (I) R₇, R₈ and R₉ are each independently hydrogen or a straight chain or branched C₂-C₄alkyl, for example a straight chain C₁-C₄ alkyl. In some embodiments, R₇, R₈ and R₉ are each independently selected hydrogen, methyl, ethyl, n-propyl, isopropyl, n-butyl and isobutyl. In other embodiments, R₇, R₈ and R₉ are each independently hydrogen, methyl, ethyl.

In formula (I) the anion, X⁻, may be any pharmaceutically acceptable anion, for example, Cl⁻, I⁻, Br⁻, ascorbate, hydrofumarate, and the like. An exemplary anion, X⁻, is iodide, I⁻. Other pharmaceutically acceptable salts may be prepared by anion exchange techniques known in the art to exchange the iodide anion for a desired pharmaceutically acceptable anion. For example, the iodide anion may be exchanged using an anion exchange resin.

Exemplary compounds of formula (I) are:

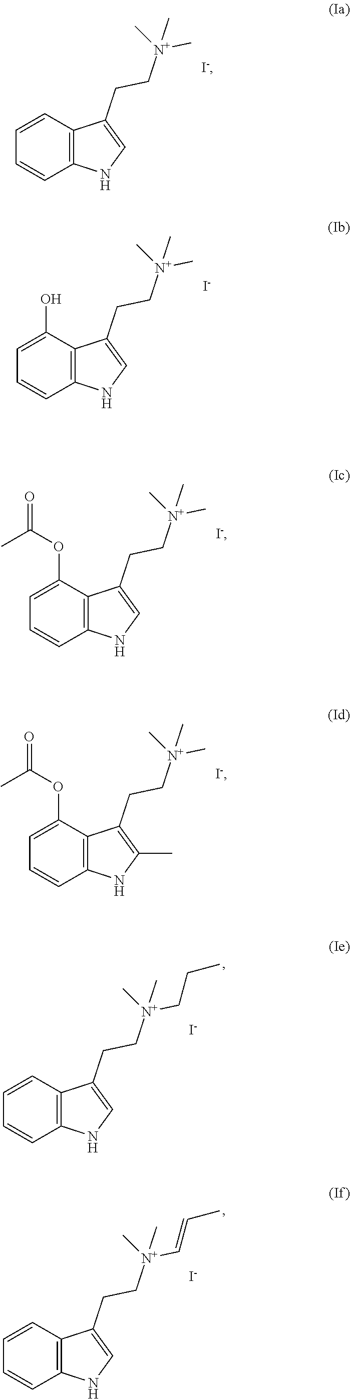

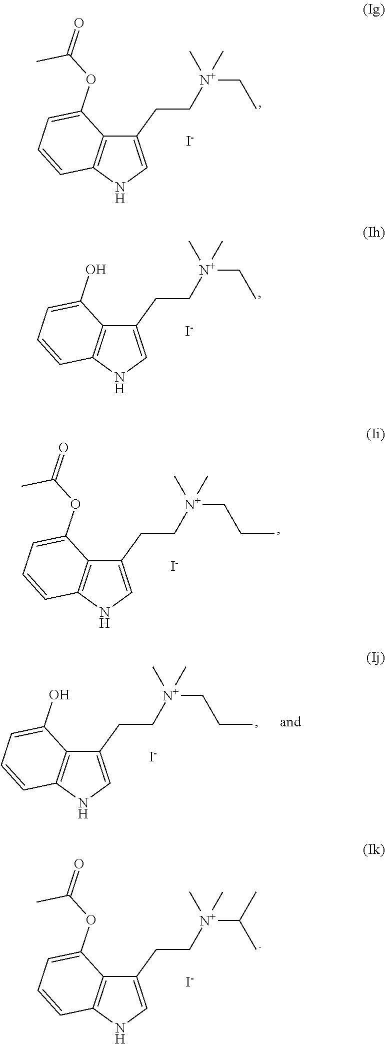

Exemplary compounds of formula (I) are those with the proviso that when R₄, R₇, R₈ and R₉ are hydrogen and R₆ is hydroxy or methoxy, R₁, R₂, and R₃ are not each methyl.

Other exemplary compounds of formula (I) are those with the proviso that when R₄, R₇, R₈ and R₉ are hydrogen, R₆ is hydroxy, and R₁ and R₂ are methyl, R₃ is not ethyl.

Other exemplary compounds of formula (I) are those where at least one of R₄, R₆, R₇, R₈, and R₉ is not hydrogen.

Other exemplary compounds of formula (I) are those where R₁ and R₂ are each independently selected from straight chain or branched C₁-C₆ alkyl or C₂-C₆ alkenyl; and R₃ is selected from a straight chain or branched C₃-C₆ alkyl or C₂-C₆ alkenyl.

Other exemplary compounds of formula (I) are those where R₆ is selected from hydrogen, C₁-C₆ alkoxy, —OC(O)R₅, and —OC(O)OR₅.

Other exemplary compounds of formula (I) are those where R₆ is —OC(O)R₅.

Other exemplary compounds of formula (I) are those where R₆ is —OC(O)OR₅.

Other exemplary compounds of formula (I) are those with the proviso that R₁, R₂ and R₃ are not all methyl when R₄ or Reis hydroxyl and R₇, R₈, and R₉ are hydrogen.

Other exemplary compounds of formula (I) are those where R₄ is selected from hydrogen, C₁-C₆ alkoxy, —OC(O)R₅, and —OC(O)OR₅.

Other exemplary compounds of formula (I) are those where R₄ is —OC(O)R₅.

Other exemplary compounds of formula (I) are those where R₄ is —OC(O)OR₅.

Other exemplary compounds of formula (I) are those where X— is selected from Cl⁻, I⁻, Br⁻ and hydrofumarate.

A compound of any of the preceding claims, wherein each of R₁, R₂ and R₃ is independently selected from methyl, ethyl, n-propyl, and isopropyl.

The disclosure also relates to a compound of formula (II):

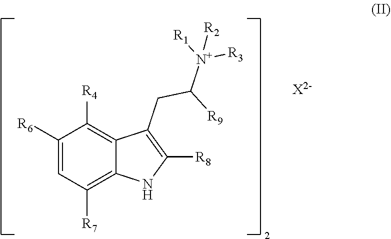

wherein
   R₁, R₂ and R₃ are independently selected from straight chain or branched C₁-C₆ alkyl or a straight chain or branched C₂-C₆ alkenyl;
   R₄ and R₆ are independently chosen from hydrogen, hydroxyl, —OR₅, —OC(O)R₅, and —OC(O)OR₅;
   R₅ is a straight chain or branched C₁-C₆ alkyl or a substituted or unsubstituted aryl; R₇, R₈ and R₉ are each independently hydrogen or a straight chain or branched C₁-C₆ alkyl; and
   X²⁻ pharmaceutically-acceptable dianion.

In formula (II) R₁, R₂ and R₃ are each independently a straight chain or branched C₁-C₆ alkyl, for example a straight chain C₁-C₆ alkyl, or a C₂-C₆ alkenyl, for example allyl. In some embodiments, R₁, R₂ and R₃ are each independently a straight chain or branched C₂-C₄ alkyl, for example a straight chain C₂-C₄ alkyl, or a C₂-C₄ alkenyl. R₁, R₂ and R₃ may each independently be selected from methyl, ethyl, n-propyl, isopropyl, n-butyl, isobutyl and tert-butyl. In other embodiments, R₁, R₂ and R₃ are each methyl, are each ethyl, or a mixture of methyl and ethyl groups.

In formula (II) R₄ and R₆ may be hydrogen, hydroxyl, —OR₅, —OC(O)R₅, and —OC(O)OR₅. When R₄ or R₆ are C₁-C₆ alkoxy group, or in some embodiments a C₁-C₄ alkoxy group, it may be a straight chain or branched C₁-C₆ alkoxy group or C₁-C₄ alkoxy group, for example a straight chain, and may be methoxy or ethoxy. R₅ is a straight chain or branched C₁-C₆ alkyl or a substituted or unsubstituted aryl. R₅ may be a straight chain or branched C₁-C₄ alkyl, for example a straight chain C₁-C₄ alkyl. In some embodiments, R₅ is selected from methyl, ethyl, n-propyl or n-butyl, and for example is methyl or ethyl. R₅ may also be a substituted or unsubstituted aryl. An aryl is a 6- to 14-membered aromatic ring, preferably a 6- to 10-membered aromatic ring and includes polycyclic ring systems in which two or more carbon atoms are common to adjoining rings where at least one ring is aromatic. Examples of aryl groups include, but are not limited to phenyl, naphthyl, anthracenyl and phenantherenyl. An aryl group may be substituted with one or more C₁-C₄ alkyl or perfluoralkyl groups, C₁-C₄ hydroxyalkyl groups, hydroxyl groups, nitro groups or halo groups (e.g. F, Cl, I or Br). An aryl group may be ortho-, meta- and/or para-substituted, preferably para-substituted. When an aryl group is substituted with one or more straight chain or branched C₁-C₄ alkyl perfluoralkyl groups the group may be methyl, trifluromethyl, ethyl, n-propyl, isopropyl, n-butyl, isobutyl or tert-butyl or the group may be methyl, ethyl, isopropyl, or tert-butyl. When R₃ or R₄ is —OR₅, —OC(O)R₅, —OC(O)OR₅, or —OSO₂R₅, R₅ may be a methyl (except when R₃ is —OC(O)R₅, R₅ is not methyl), a tert-butyl, a phenyl, a benzyl, a para-halophenyl or a para-tolyl group.

In formula (II) R₇, R₈ and R₉ are each independently hydrogen or a straight chain or branched C₁-C₄ alkyl, for example a straight chain C₁-C₄ alkyl. In some embodiments, R₇, R₈ and R₉ are each independently selected hydrogen, methyl, ethyl, n-propyl, isopropyl, n-butyl and isobutyl. In other embodiments, R₇, R₈ and R₉ are each independently hydrogen, methyl, ethyl.

In formula (II) the anion, X²⁻, may be any pharmaceutically acceptable dianion, e.g., fumarate, maleate, malonate, succinate, tartarate, or oxalate, and the like. Other pharmaceutically acceptable salts may be prepared by anion exchange techniques known in the art to exchange the iodide anion for a desired pharmaceutically acceptable anion. For example, the iodide anion may be exchanged using an anion exchange resin.

Exemplary compounds of formula (II) are those where one of R₄ and R₆ is hydrogen and the other of R₄ and Reis selected from —OC(O)OR₅ and —OC(O)R₅.

Other exemplary compounds of formula (II) are those where one of R₄ and R₆ is hydrogen and the other of R₄ and R₆ is selected from —OC(O)R₅.

Other exemplary compounds of formula (II) are those where one of R₄ and R₆ is hydrogen and the other of R₄ and R₆ is selected from —OC(O)OR₅.

Other exemplary compounds of formula (II) are those where one of R₄ and R₆ is hydrogen and the other of R₄ and R₆ is selected from —OR₅.

Other exemplary compounds of formula (II) are those where R₅ is methyl.

Other exemplary compounds of formula (II) are those where R₅ is ethyl.

Other exemplary compounds of formula (II) are those where R₅ is selected from straight chain or branched C₃-C₆ alkyl.

Other exemplary compounds of formula (II) are those where R₁, R₂ and R₃ are independently selected from methyl, ethyl, n-propyl, and isopropyl.

Other exemplary compounds of formula (II) are those where X²⁻ is selected from fumarate, malonate, succinate, tartarate, oxalate, and maleate.

The disclosure also relates to a zwitterionic compound of formula (III):

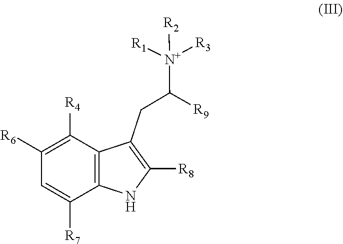

wherein
   R₁, R₂ and R₃ are each independently a straight chain or branched C₁-C₆ alkyl or C₂-C₆ alkenyl;
   R₄ is hydrogen, —O⁻, C₁-C₆ alkoxy, —OC(O)R₅, —OC(O)O⁻, —OSO₂O⁻, or —OP(O)OHO⁻;
   R₅ is a straight chain or branched C₁-C₆ alkyl;
   R₆ is hydrogen, —O⁻, C₁-C₆ alkoxy, —OC(O)R₅, —OC(O)OR₅, or a straight chain or branched C₁-C₆ alkyl; and
   R₇, R₈ and R₉ are each independently hydrogen or a straight chain or branched C₁-C₆ alkyl,
wherein at least one of R₄ or R₆ is —O⁻, —OC(O)O⁻, —OSO₂O⁻, or —OP(O)OHO⁻, provided that R₄ is not —OP(O)OHO⁻ when R₁, R₂ and R₃ are each methyl.

In formula (III) R₁, R₂ and R₃ are each independently a straight chain or branched C₁-C₆ alkyl, for example a straight chain C₁-C₆ alkyl, or a C₂-C₆ alkenyl, for example allyl. In some embodiments, R₁, R₂ and R₃ are each independently a straight chain or branched C₁-C₄ alkyl, for example a straight chain C₁-C₄ alkyl, or a C₂-C₄ alkenyl. R₁, R₂ and R₃ may each independently be selected from methyl, ethyl, n-propyl, isopropyl, n-butyl, isobutyl and tert-butyl. In other embodiments, R₁, R₂ and R₃ are each methyl, are each ethyl, or a mixture of methyl and ethyl groups.

In formula (III) R₄ may be hydrogen, —O⁻, C₁-C₆ alkoxy, —OC(O)R₅, —OC(O)O⁻, —OSO₂O⁻, or —OP(O)OHO⁻. When R₄ is C₁-C₆ alkoxy group, or in some embodiments a C₁-C₄ alkoxy group, it may be a straight chain or branched C₁-C₆ alkoxy group or C₁-C₄ alkoxy group, for example a straight chain, and may be methoxy or ethoxy. R₅ is a straight chain or branched C₁-C₆ alkyl. R₅ may be a straight chain or branched C₂-C₄ alkyl, for example a straight chain CᵣC₄ alkyl. In some embodiments, R₅ is selected from methyl, ethyl, n-propyl or n-butyl, and for example is methyl or ethyl.

In formula (III) R₆ may be hydrogen, —O⁻, C₁-C₆ alkoxy, —OC(O)R₅, —OC(O)OR₅, or a straight chain or branched C₁-C₆ alkyl. When R₆ is C₁-C₆ alkoxy group, or in some embodiments a C₁-C₄ alkoxy group, it may be a straight chain or branched C₁-C₆ alkoxy group or C₁-C₄ alkoxy group, for example a straight chain, and may be methoxy or ethoxy. R₅ is a straight chain or branched C₁-C₆ alkyl.

In formula (III) R₇, R₈ and R₉ are each independently hydrogen or a straight chain or branched C₁-C₄ alkyl, for example a straight chain C₁-C₄ alkyl. In some embodiments, R₇, R₈ and R₉ are each independently selected hydrogen, methyl, ethyl, n-propyl, isopropyl, n-butyl and isobutyl. In other embodiments, R₆, R₇, R₈ and R₉ are each independently hydrogen, methyl, ethyl.

Exemplary compounds of formula (III) are those where R₆ is not —O⁻ when R₁, R₂ and R₃ are methyl, R₄ and R₇ are hydrogen, R₈ is methyl, and R₉ is hydrogen.

Other exemplary compounds of formula (III) are those where R₁, R₂ and R₃ are selected from methyl, ethyl, n-propyl, and isopropyl.

Other exemplary compounds of formula (III) are those where R₄ and R₆ are selected from hydrogen and —O⁻.

Other exemplary compounds of formula (III) are those where R₇ is hydrogen.

Other exemplary compounds of formula (III) are those where R₈ is hydrogen.

Other exemplary compounds of formula (III) are those where R₉ is hydrogen.

Other exemplary compounds of formula (III) are those where one of R₄ and R₆ is hydrogen, and the other of R₄ and R₆ is selected from —O⁻, —OC(O)O⁻, —OSO₂O⁻, or —OP(O)OHO⁻.

Other exemplary compounds of formula (III) are those where one of R₄ and R₆ is hydrogen, and the other of R₄ and R₆ is —O⁻.

Other exemplary compounds of formula (III) are those where one of R₄ and R₆ is hydrogen, and the other of R₄ and R₆ is —OC(O)O⁻.

Other exemplary compounds of formula (III) are those where one of R₄ and R₆ is hydrogen, and the other of R₄ and R₆ is —OSO₂O⁻.

Other exemplary compounds of formula (III) are those where one of R₄ and R₆ is hydrogen, and the other of R₄ and R₆ is —OP(O)OHO⁻.

A compound of formula (I), formula (II), or formula (III) may be prepared by reacting a compound of formula (Ia) with XR₃, where X is I, by refluxing in an appropriate organic solvent, such as methanol or THF, under an inert atmosphere.

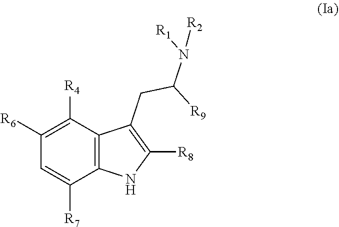

In a preferred embodiment, XR₃ is ICH₃ or ICH₂CH₃. When R₄ or R₆ are hydroxyl they can be converted to esters, —OC(O)R₅, or carbonate esters, —OC(O)OR₅, as is known in the art. Hydroxyl groups may be introduced by hydrolysis of a corresponding ester. Other pharmaceutically acceptable salts may be prepared by anion exchange techniques known in the art to exchange the iodide anion for a desired pharmaceutically acceptable anion. For example, the iodide anion may be exchanged using an anion exchange resin.

Methods of Treatment and Therapeutic Uses

Compounds of formula (I), formula (II), or formula (III) according to the disclosure, crystalline forms thereof, and the methods and the compositions (e.g., pharmaceutical compositions) are used to regulate the activity of a neurotransmitter receptor by administering a therapeutically effective dose of compounds of formula (I), formula (II), or formula (III) according to the disclosure, and the methods and the compositions (e.g., pharmaceutical compositions) are used to treat inflammation and/or pain by administering a therapeutically effective dose of compounds of formula (I), formula (II), or formula (III) according to the disclosure.

Methods of the disclosure also related to the administration of a therapeutically effective amount of compounds of formula (I), formula (II), or formula (III) according to the disclosure to prevent or treat a disease or condition, such as those discussed below for a subject in need of treatment. Compounds of formula (I), formula (II), or formula (III) according to the disclosure may be administered neat or as a composition comprising compounds of formula (I), formula (II), or formula (III) according to the disclosure as discussed below.

Compounds of formula (I), formula (II), or formula (III) according to the disclosure may be used to prevent and/or treat a psychological disorder. The disclosure provides a method for preventing and/or treating a psychological disorder by administering to a subject in need thereof a therapeutically effective amount of compounds of formula (I), formula (II), or formula (III) according to the disclosure, including the exemplary embodiments discussed herein. The psychological disorder may be chosen from depression, psychotic disorder, schizophrenia, schizophreniform disorder (acute schizophrenic episode); schizoaffective disorder; bipolar I disorder (mania, manic disorder, manic-depressive psychosis); bipolar II disorder; major depressive disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder; anxiety disorder; social anxiety disorder; substance-induced anxiety disorder; selective mutism; panic disorder; panic attacks; agoraphobia; attention deficit syndrome, post-traumatic stress disorder (PTSD), premenstrual dysphoric disorder (PMDD), and premenstrual syndrome (PMS).

Compounds of formula (I), formula (II), or formula (III) according to the disclosure may be used to prevent and/or treat a brain disorder. The disclosure provides a method for preventing and/or treating a brain disorder (e.g., Huntington's disease, Alzheimer's disease, dementia, and Parkinson's disease) by administering to a subject in need thereof a therapeutically effective amount of compounds of formula (I), formula (II), or formula (III) according to the disclosure, including the exemplary embodiments discussed above.

Compounds of formula (I), formula (II), or formula (III) according to the disclosure may be used to prevent and/or treat developmental disorders, delirium, dementia, amnestic disorders and other cognitive disorders, psychiatric disorders due to a somatic condition, drug-related disorders, schizophrenia and other psychotic disorders, mood disorders, anxiety disorders, somatoform disorders, factitious disorders, dissociative disorders, eating disorders, sleep disorders, impulse control disorders, adjustment disorders, or personality disorders. The disclosure provides a method for preventing and/or treating these disorders by administering to a subject in need thereof a therapeutically effective amount of compounds of formula (I), formula (II), or formula (III) according to the disclosure, including the exemplary embodiments discussed above.

Compounds of formula (I), formula (II), or formula (III) according to the disclosure may be used to prevent and/or treat inflammation and/or pain, such as for example inflammation and/or pain associated with inflammatory skeletal or muscular diseases or conditions. The disclosure provides a method for preventing and/or treating an inflammation and/or pain by administering to a subject in need thereof a therapeutically effective amount of compounds of formula (I), formula (II), or formula (III) according to the disclosure, including the exemplary embodiments discussed herein. Generally speaking, treatable “pain” includes nociceptive, neuropathic, and mix-type. A method of the disclosure may reduce or alleviate the symptoms associated with inflammation, including but not limited to treating localized manifestation of inflammation characterized by acute or chronic swelling, pain, redness, increased temperature, or loss of function in some cases. A method of the disclosure may reduce or alleviate the symptoms of pain regardless of the cause of the pain, including but not limited to reducing pain of varying severity, i.e., mild, moderate and severe pain, acute pain and chronic pain. A method of the disclosure is effective in treating joint pain, muscle pain, tendon pain, burn pain, and pain caused by inflammation such as rheumatoid arthritis. Skeletal or muscular diseases or conditions which may be treated include but are not limited to musculoskeletal sprains, musculoskeletal strains, tendinopathy, peripheral radiculopathy, osteoarthritis, joint degenerative disease, polymyalgia rheumatica, juvenile arthritis, gout, ankylosing spondylitis, psoriatic arthritis, systemic lupus erythematosus, costochondritis, tendonitis, bursitis, such as the common lateral epicondylitis (tennis elbow), medial epicondylitis (pitchers elbow) and trochanteric bursitis, temporomandibular joint syndrome, and fibromyalgia.

Compounds of formula (I), formula (II), or formula (III) according to the disclosure may be used to modulate activity of a mitogen activating protein (MAP), comprising administering a composition of the disclosure. In one embodiment, the mitogen activating protein (MAP) comprises a MAP kinase (MAPk). MAPKs provide a wide-ranging signaling cascade that allow cells to quickly respond to biotic and abiotic stimuli. Exemplary MAPKs include, but are not limited to, Tropomyosin Receptor Kinase A (TrkA), P38-alpha, Janus Kinase 1 (JAK1), and c-Jun N-Terminal Kinase 3 (JNK3). TrkA is a high affinity catalytic receptor of nerve growth factor (NGF) protein. TrkA regulates NGF response, influencing neuronal differentiation and outgrowth as well as programmed cell death. p38-alpha is involved with the regulation of pro-inflammatory cytokines, including TNF-α. In the central nervous system, p38-alpha regulates neuronal death and neurite degeneration, and it is a common target of Alzheimer's disease therapies. JAK1 influences cytokine signaling, including IL-2, IL-4, IFN-alpha/beta, IFN-γ, and IL-10, and it is implicated in brain aging. JNK3 is neuronal specific protein isoform of the JNKs. It is involved with the regulation of apoptosis. JNK3 also plays a role in modulating the response of cytokines, growth factors, and oxidative stress.

As used herein, the term “modulating activity of a mitogen activating protein” refers to changing, manipulating, and/or adjusting the activity of a mitogen activating protein. In one embodiment, modulating the activity of a MAP, such as a MAPK, can influence neural health, neurogenesis, neural growth and differentiation, and neurodegenerative diseases.

Compounds of formula (I), formula (II), or formula (III) according to the disclosure may be used to modulate neurogenesis, comprising administering a composition of the disclosure. As used herein, the term “modulating neurite outgrowth” refers to changing, manipulating, and/or adjusting the growth and development of neural projections, or “neurites.” In one embodiment, neurogenesis comprises modulating the growth of new neurites, the number of neurites per neuron, and/or neurite length. In one embodiment, modulating neurite outgrowth comprises increasing and/or enhancing the rate and/or length at which neurites develop.

Compounds of formula (I), formula (II), or formula (III) according to the disclosure may be used to modulate neurite outgrowth, comprising administering a composition of the disclosure. As used herein, the term “modulating neurogenesis” refers to changing, manipulating, and/or adjusting the growth and development of neural tissue. In one embodiment, neurogenesis comprises adult neurogenesis, in which new neural stem cells are generated from neural stem cells in an adult animal. In one embodiment, modulating neurogenesis comprises increasing and/or enhancing the rate at which new neural tissue is developed.

The disclosure also relates to a method of generating a dialkyltryptamine compound in situ in a patient, the method comprising administering at least one quaternary tryptamine compound selected from formulas (I), (II) and (III) to the patient. The disclosure also relates to methods of generating a dialkyltryptamine compound comprising contacting at least one quaternary tryptamine compound selected from formulas (I), (II) and (III) with an enzyme in vitro or in vivo, such as an enzyme capable of nitrogen dealkylation (e.g., cytochrome P450 enzymes (CYPs)). The disclosure further relates to methods of generating a dialkyltryptamine compound in situ in a patient comprising contacting at least one quaternary tryptamine compound selected from formulas (I), (II) and (III) with an enzyme in the patient capable of nitrogen dealkylation (e.g., cytochrome P450 enzymes (CYPs)).

Other exemplary compounds of formula (I), formula (II), or formula (III) are:
7-methyl-N,N,N-triethyltryptamine (7-Me-TET) Iodide
7-methyl-N,N,N-trimethyltryptamine (7-Me-TMT) Iodide
1-methyl-N,N,N-trimethyltryptamine (1-Me-TMT) iodide
5-methyl-N,N,N-tri-n-propyltryptamine (5-Me-TPT) Iodide Acetonitrile
5-methoxy-N,N,N-triethyltryptamine (5-MeO-TET) Iodide
5-methoxy-N,N,N-trimethyltryptamine (5-MeO-TMT) Iodide
5-methoxy-N,N,N-tri-n-propyltryptamine (5-MeO-TPT) Iodide
5-hydroxy-N,N,N-tri-n-propyltryptamine (5-HO-TPT) Iodide
5-hydroxy-N,N,N-triethyltryptamine 5-HO-TET Iodide
N-methyl-N,N-di-n-propyltryptamine (MDPT) Iodide
N,N,N-tri-n-propyltryptamine (TPT) Iodide
N,N,N-triethyltryptamine (TET) Iodide
5-methyl-N,N,N-trimethyltryptamine (5-Me-TMT) Iodide
5-methyl-N,N,N-trimethyltryptamine (5-Me-TMT) Iodide Isopropanol
5-methoxy-N-methyl-N,N-di-n-propyltryptamine (5-MeO-MDPT) Iodide
5-methoxy-2-methyl-N,N,N-tri-n-propyltryptamine (5-MeO-2-Me-TPT) Iodide
5-methoxy-2-methyl-N,N,N-triethyltryptamine (5-MeO-2-Me-TET) Iodide Hydrate
N-methyl-N-n-propyl-N-n-butyltryptamine (MPBT) Iodide
N-methyl-N-ethyl-N-n-propyltryptamine (MEPT) Iodide
4-hydroxy-N,N-dimethyl-N-allyltryptamine (4-HO-DMALT) Iodide
4-hydroxy-N,N-dimethyl-N-isopropyltryptamine (4-HO-DMiPT) Iodide
4-acetoxy-N,N-dimethyl-N-n-butyltryptamine (4-AcO-DMBT) Iodide
4-acetoxy-N,N-dimethyl-N-allyltryptamine (4-AcO-DMALT) Iodide

Compositions

The disclosure also relates to compositions comprising an effective amount of a compound of formula (I), formula (II), or formula (III) according to the disclosure (alkyl quaternary tryptamine compounds of the disclosure), including its exemplary embodiments discussed above, and an excipient (e.g., a pharmaceutically-acceptable excipient). In another embodiment, the disclosure also relates to pharmaceutical compositions comprising a therapeutically effective amount of alkyl quaternary tryptamine compounds of the disclosure, including their exemplary embodiments discussed above, and a pharmaceutically acceptable excipient (also known as a pharmaceutically acceptable carrier). As discussed above, an alkyl quaternary tryptamine compound of the disclosure may be, for example, therapeutically useful to prevent and/or treat the psychological disorders, brain disorders, pain, and inflammation as well as the other disorders described herein.

A composition or a pharmaceutical composition of the disclosure may be in any form which contains an alkyl quaternary tryptamine compound of the disclosure. The composition may be, for example, a tablet, capsule, liquid suspension, injectable, topical, or transdermal. The compositions generally contain, for example, about 1% to about 99% by weight of an alkyl quaternary tryptamine compound of the disclosure and, for example, 99% to 1% by weight of at least one suitable pharmaceutically acceptable excipient. In one embodiment, the composition may be between about 5% and about 75% by weight of an alkyl quaternary tryptamine compound of the disclosure, with the rest being at least one suitable pharmaceutically acceptable excipient or at least one other adjuvant, as discussed below.

Published US applications US 2018/0221396 A1 and US 2019/0142851 A1 disclose compositions comprising a combination of a first purified psilocybin derivative with a second purified psilocybin derivative, with one or two purified cannabinoids or with a purified terpene. Various ratios of these components in the composition are also disclosed. The disclosures of US 2018/0221396 A1 and US 2019/0142851A1 are incorporated herein by reference. According to this disclosure, a compound of formula (I), formula (II), or formula (III) of the disclosure may be used as the “first purified psilocybin derivative” in the compositions described in US 2018/0221396 A1 and US 2019/0142851 A1. Accordingly, this disclosure provides a composition comprising: a first component comprising at least one alkyl quaternary tryptamine compound of the disclosure; at least one second component selected from at least one of (a) a serotonergic drug, (b) a purified psilocybin derivative, (c) a purified cannabinoid or (d) a purified terpene; and at least one pharmaceutically-acceptable excipient or at least one other adjuvant. Such a composition may be a pharmaceutical composition wherein the components are present individually in therapeutically effective amounts or by combination in a therapeutically effective amount to treat a disease, disorder, or condition as described herein.

When used in such compositions as a first component comprising at least one alkyl quaternary tryptamine compound of the disclosure with a second component selected from at least one of (a) a serotonergic drug, (b) a purified psilocybin derivative, (c) a purified cannabinoid, or (d) a purified terpene, the compositions represent particular embodiments of the disclosure. Compositions having as a first component at least one alkyl quaternary tryptamine compound of the disclosure with a second component selected from at least one of (e) an adrenergic drug, (f) a dopaminergic drug, (g) a monoamine oxidase inhibitor, (h) a purified erinacine, or (i) a purified hericenone, also represent additional particular embodiments of the disclosure represented by the compositions having the alkyl quaternary tryptamine compound of the disclosure. In some embodiments, the first and second components can be administered at the same time (e.g., together in the same composition), or at separate times over the course of treating a patient in need thereof. Such a composition may be a pharmaceutical composition wherein the components are present individually in therapeutically effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments, incorporated here by reference. Exemplary psilocybin derivatives include but are not limited to psilocybin itself and the psilocybin derivates described in paragraphs [0081]-[0109] of US 2018/0221396 A1 and [082]-[0110] US 2019/0142851 A1 as well as the disclosed exemplary embodiments. Exemplary cannabinoids include but are not limited to the cannabinoids described in paragraphs [0111]-[0159] of US 2018/0221396 A1 and [0112]-[0160] US 2019/0142851 A1 as well as the disclosed exemplary embodiments. Exemplary terpenes include but are not limited to the terpenes described in paragraphs [0160]-[0238] of US 2018/0221396 A1 and [0161]-[0300] US 2019/0142851 A1 as well as the disclosed exemplary embodiments.

A pharmaceutical formulation of the disclosure may comprise, consist essentially of, or consist of (a) at least one alkyl quaternary tryptamine compound of the disclosure and (b) at least one second active compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone and (c) a pharmaceutically acceptable excipient. In some embodiments, the alkyl quaternary tryptamine compound(s) of the disclosure and the second active compound(s) are each present in a therapeutically effective amount using a purposefully engineered and unnaturally occurring molar ratios. Exemplary molar ratios of the alkyl quaternary tryptamine compounds of the disclosure to the second active compound in a composition of the disclosure include but are not limited to from about 0.1:100 to about 100:0.1, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1 or may be about 1:1.

A pharmaceutical formulation of the disclosure may comprise a composition containing an alkyl quaternary tryptamine compound of the disclosure and a serotonergic drug, a purified psilocybin derivative, a purified cannabinoid, or a purified terpene, each present in a therapeutically effective amount using a purposefully engineered and unnaturally occurring molar ratios. Published US applications US 2018/0221396 A1 and US 2019/0142851 A1 disclose compositions comprising a combination of a purified psilocybin derivative with a second purified psilocybin derivative, with one or two purified cannabinoids or with a purified terpene. The disclosures of US 2018/0221396 A1 and US 2019/0142851A1 are incorporated herein by reference. According to this disclosure composition containing an alkyl quaternary tryptamine compound of the disclosure may be used in place of a “purified psilocybin derivative” in the compositions described in US 2018/0221396 A1 and US 2019/0142851 A1. Accordingly, the disclosure provides a pharmaceutical formulation comprising as (a) at least one alkyl quaternary tryptamine compound of the disclosure and at least one second component selected from (b) a purified psilocybin derivative, (c) a purified cannabinoid or (d) a purified terpene; and at least one pharmaceutically-acceptable excipient or at least one other adjuvant, as described herein. Such a composition may be a pharmaceutical composition wherein the components are present individually in therapeutic effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments, incorporated here by reference. Some exemplary serotonergic drugs include SSRIs and SNRIs. Some examples of specific serotonergic drugs include the following molecules, including any salts, solvates, or polymorphs thereof: 6-Allyl-N,N-diethyl-NL, N,N-Dibutyl-T, N,N-Diethyl-T, N,N-Diisopropyl-T, 5-Methyoxy-alpha-methyl-T, N,N-Dimethyl-T, 2,alpha-Dimethyl-T, alpha,N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C₁₋₇-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetramethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N,N-dimethyl-T, N-Isopropyl-4-methoxy-N-methyl-T, N-Isopropyl-5-methoxy-N-methyl-T, 5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C₁₋₅-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, and 7-Methoxy-1-methyl-1,2,3,4-tetrahydro-C, alpha,N-Dimethyl-5-methoxy-T. For additional information regarding these compounds see Shulgin, A. T., & Shulgin, A. (2016). Tihkal: The Continuation. Berkeley, Calif.: Transform Press. In one embodiment, a serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and/or derivatives thereof. In an exemplary embodiment, the serotonergic drug is 3,4-methylenedioxymethamphetamine.

Exemplary psilocybin derivatives include but are not limited to psilocybin itself and the psilocybin derivates described in paragraphs [0081]-[0109] of US 2018/0221396 A1 and [082]-[0110] US 2019/0142851A1 as well as the disclosed exemplary embodiments, incorporated here by reference. In one embodiment, the compositions disclosed herein comprise one or more purified psilocybin derivatives chosen from: [3-(2-Dimethylaminoethyl)-1H-indol-4-yl] dihydrogen phosphate, 4-hydroxytryptamine, 4-hydroxy-N,N-dimethyltryptamine, [3-(2-methylaminoethyl)-1H-indol-4-yl]dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine.

Exemplary cannabinoids include but are not limited to the cannabinoids described in paragraphs [0111]-[0159] of US 2018/0221396 A1 and [0112]-[0160] US 2019/0142851 A1 as well as the disclosed exemplary embodiments, incorporated here by reference. Examples of cannabinoids within the context of this disclosure include the following molecules: Cannabichromene (CBC), Cannabichromenic acid (CBCA), Cannabichromevarin (CBCV), Cannabichromevarinic acid (CBCVA), Cannabicyclol (CBL), Cannabicyclolic acid (CBLA), Cannabicyclovarin (CBLV), Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoic acid B (CBEA-B), Cannabielsoin (CBE), Cannabielsoin acid A (CBEA-A), Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA), Cannabinodiol (CBND), Cannabinodivarin (CBDV), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV), Cannabitriol (CBT), Cannabitriolvarin (CBTV), 10-Ethoxy-9-hydroxy-delta-6a-tetrahydrocannabinol, Cannbicitran (CBT), Cannabiripsol (CBR), 8,9-Dihydroxy-delta-6a-tetrahydrocannabinol, Delta-8-tetrahydrocannabinol (Δ8-THC), Delta-8-tetrahydrocannabinolic acid (Δ8-THCA), Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA), 10-Oxo-delta-6a-tetrahydrocannabinol (OTHC), Cannabichromanon (CBCF), Cannabifuran (CBF), Cannabiglendol, Delta-9-cis-tetrahydrocannabinol (cis-TFIC), Tryhydroxy-delta-9-tetrahydrocannabinol (triOH-THC), Dehydrocannabifuran (DCBF), and 3,4,5,6-Tetrahydro-7-hydroxy-alpha-alpha-2-trimethyl-9-n-propyl-2,6-metha-no-2H-1-benzoxocin-5-methanol. In one embodiment, the purified cannabinoid is chosen from TFIC, TFICA, TFICV, TFICVA, CBC, CBCA, CBCV, CBCVA, CBD, CBDA, CBDV, CBDVA, CBG, CBGA, CBGV, or CBGVA.

Exemplary terpenes include but are not limited to the terpenes described in paragraphs [0160]-[0238] of US 2018/0221396 A1 and [0161]-[0300] US 2019/0142851 A1 as well as the disclosed exemplary embodiments, incorporated here by reference. In one embodiment, a purified terpene is chosen from acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, caryophyllene, caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1(10),11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, or vanillin. In one embodiment, a purified terpene is chosen from bornyl acetate, alpha-bisabolol, borneol, camphene, camphor, carene, caryophyllene, cedrene, cymene, elemene, eucalyptol, eudesmol, farnesene, fenchol, geraniol, guaiacol, humulene, isoborneol, limonene, linalool, menthol, myrcene, nerolidol, ocimene, phellandrene, phytol, pinene, pulegone, sabinene, terpineol, terpinolene, or valencene.

As used herein, the term “adrenergic drug” refers to a compound that binds, blocks, or otherwise influences (e.g., via an allosteric reaction) activity at an adrenergic receptor. In one embodiment, an adrenergic drug binds to an adrenergic receptor. In one embodiment, an adrenergic drug indirectly affects an adrenergic receptor, e.g., via interactions affecting the reactivity of other molecules at the adrenergic receptor. In one embodiment, an adrenergic drug is an agonist, e.g., a compound activating an adrenergic receptor. In one embodiment, an adrenergic drug is an antagonist, e.g., a compound binding but not activating an adrenergic receptor, e.g., blocking a receptor. In one embodiment, an adrenergic drug is an effector molecule, e.g., a compound binding to an enzyme for allosteric regulation. In one embodiment, an adrenergic drug acts (either directly or indirectly) at more than one type of receptor (e.g., 5HT, dopamine, adrenergic, acetylcholine, etc.).

In one embodiment, an adrenergic drug is an antidepressant. In one embodiment, an adrenergic drug is a norepinephrine transporter inhibitor. In one embodiment, an adrenergic drug is a vesicular monoamine transporter inhibitor. In one embodiment, an adrenergic drug is chosen from adrenaline, agmatine, amoxapine, aptazapine, atomoxetine, bupropion, clonidine, doxepin, duloxetine, esmirtazpine, mianserin, ketanserin, mirabegron, mirtazapine, norepinephrine, phentolamine, phenylephrine, piperoxan, reserpine, ritodrine, setiptiline, tesofensine, timolol, trazodone, trimipramine, orxylazine.

As used herein, the term “dopaminergic drug” refers to a compound that binds, blocks, or otherwise influences (e.g., via an allosteric reaction) activity at a dopamine receptor. In one embodiment, a dopaminergic drug binds to a dopamine receptor. In one embodiment, a dopaminergic drug indirectly affects a dopamine receptor, e.g., via interactions affecting the reactivity of other molecules at the dopamine receptor. In one embodiment, a dopaminergic drug is an agonist, e.g., a compound activating a dopamine receptor. In one embodiment, a dopaminergic drug is an antagonist, e.g., a compound binding but not activating a dopamine receptor, e.g., blocking a receptor. In one embodiment, a dopaminergic drug is an effector molecule, e.g., a compound binding to an enzyme for allosteric regulation. In one embodiment, a dopaminergic drug acts (either directly or indirectly) at more than one type of receptor (e.g., 5HT, dopamine, adrenergic, acetylcholine, etc.).

In one embodiment, a dopaminergic drug is a dopamine transporter inhibitor. In one embodiment, a dopaminergic drug is a vesicular monoamine transporter inhibitor. In one embodiment, a dopaminergic drug is chosen from amineptine, apomorphine, benzylpiperazine, bromocriptine, cabergoline, chlorpromazine, clozapine, dihydrexidine, domperidone, dopamine, fluphenazine, haloperidol, ketamine, loxapine, methamphetamine, olanzapine, pemoline, perphenazine, pergolide, phencyclidine, phenethylamine, phenmetrazine, pimozide, piribedil, a psychostimulant, reserpine, risperidone, ropinirole, tetrabenazine, or thioridazine.

As used herein, the term “monoamine oxidase inhibitor” (MAOI) refers to a compound that blocks the actions of monoamine oxidase enzymes. In on embodiment, a MAOI inhibits the activity of one or both monoamine oxidase A and monoamine oxidase B. In one embodiment a MAOI is a reversible inhibitors of monoamine oxidase A. In one embodiment a MAOI is a drug chosen from isocarboxazid, phenelzine, or tranylcypromine.

In one embodiment, the compositions and methods disclosed herein include one or more purified erinacine molecules. In one embodiment, the compositions and methods disclosed herein comprise purified erinacine A. In one embodiment, the compositions and methods disclosed herein comprise erinacine B. In one embodiment, the compositions and methods disclosed herein comprise erinacine C. In one embodiment, the compositions and methods disclosed herein comprise erinacine D. In one embodiment, the compositions and methods disclosed herein comprise erinacine E. In one embodiment, the compositions and methods disclosed herein comprise erinacine F. In one embodiment, the compositions and methods disclosed herein comprise erinacine G. In one embodiment, the compositions and methods disclosed herein comprise erinacine H. In one embodiment, the compositions and methods disclosed herein comprise erinacine I. In one embodiment, the compositions and methods disclosed herein comprise erinacine J. In one embodiment, the compositions and methods disclosed herein comprise erinacine K In one embodiment, the compositions and methods disclosed herein comprise erinacine P. In one embodiment, the compositions and methods disclosed herein comprise erinacine Q. In one embodiment, the compositions and methods disclosed herein comprise erinacine R. In one embodiment, the compositions and methods disclosed herein comprise erinacine S.

In one embodiment, the compositions and methods disclosed herein include one or more purified hericenone molecules. In one embodiment, the compositions and methods disclosed herein comprise purified hericenone A. In one embodiment, the compositions and methods disclosed herein comprise purified hericenone B. In one embodiment, the compositions and methods disclosed herein comprise purified hericenone C. In one embodiment, the compositions and methods disclosed herein comprise purified hericenone D. In one embodiment, the compositions and methods disclosed herein comprise purified hericenone E. In one embodiment, the compositions and methods disclosed herein comprise purified hericenone F. In one embodiment, the compositions and methods disclosed herein comprise purified hericenone G. In one embodiment, the compositions and methods disclosed herein comprise purified hericenone H.

Exemplary compositions of an alkyl quaternary tryptamine compounds of the disclosure and a second compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone in exemplary molar ratios are shown in Table 1. An alkyl quaternary tryptamine compound of the disclosure may be any one of the exemplary embodiments described above including their crystalline forms as disclosed herein.

TABLE 1

| | Molar ratio of an | Molar ratio of an | Molar ratio of an |
|---|---|---|---|
| | alkyl quaternary | alkyl quaternary | alkyl quaternary |
| | tryptamine | tryptamine | tryptamine |
| | compound:second | compound:second | compound:second |
| Second Compound | compound | compound | compound |
| 3,4- | About 1:100 to | About 1:25 to | About 1:5 to |
| methylenedioxymethamphetamine | about 100:1 | about 25:1 | about 5:1 |
| Citalopram | About 1:100 to | About 1:25 to | About 1:5 to |
| | about 100:1 | about 25:1 | about 5:1 |
| Escitalopram | About 1:100 to | About 1:25 to | About 1:5 to |
| | about 100:1 | about 25:1 | about 5:1 |
| Fluoxetine | About 1:100 to | About 1:25 to | About 1:5 to |
| | about 100:1 | about 25:1 | about 5:1 |
| Paroxetine | About 1:100 to | About 1:25 to | About 1:5 to |
| | about 100:1 | about 25:1 | about 5:1 |
| Sertraline | About 1:100 to | About 1:25 to | About 1:5 to |
| | about 100:1 | about 25:1 | about 5:1 |
| [3-(2-Dimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxytryptamine | About 1:100 to | About 1:25 to | About 1:5 to |
| | about 100:1 | about 25:1 | about 5:1 |
| 4-hydroxy-N,N-dimethyltryptamine | About 1:100 to | About 1:25 to | About 1:5 to |
| | about 100:1 | about 25:1 | about 5:1 |
| [3-(2-methylaminoethyl)-1H-indol- | About 1:100 to | About 1:25 to | About 1:5 to |
| 4-yl] dihydrogen phosphate | about 100:1 | about 25:1 | about 5:1 |
| 4-hydroxy-N-methyltryptamine | About 1:100 to | About 1:25 to | About 1:5 to |
| | about 100:1 | about 25:1 | about 5:1 |
| [3-(aminoethyl)-1H-indol-4-yl] | About 1:100 to | About 1:25 to | About 1:5 to |
| dihydrogen phosphate | about 100:1 | about 25:1 | about 5:1 |
| [3-(2-trimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxy-N,N,N- | About 1:100 to | About 1:25 to | About 1:5 to |
| trimethyltryptamine | about 100:1 | about 25:1 | about 5:1 |
| THC | About 1:100 to | About 1:25 to | About 1:5 to |
| | about 100:1 | about 25:1 | about 5:1 |
| CBC | About 1:100 to | About 1:25 to | About 1:5 to |
| | about 100:1 | about 25:1 | about 5:1 |
| CBD | About 1:100 to | About 1:25 to | About 1:5 to |
| | about 100:1 | about 25:1 | about 5:1 |
| CBG | About 1:100 to | About 1:25 to | About 1:5 to |
| | about 100:1 | about 25:1 | about 5:1 |
| Myrcene | About 1:100 to | About 1:25 to | About 1:5 to |
| | about 100:1 | about 25:1 | about 5:1 |
| Pinene | About 1:100 to | About 1:25 to | About 1:5 to |
| | about 100:1 | about 25:1 | about 5:1 |
| Caryophyllene | About 1:100 to | About 1:25 to | About 1:5 to |
| | about 100:1 | about 25:1 | about 5:1 |
| Limonene | About 1:100 to | About 1:25 to | About 1:5 to |
| | about 100:1 | about 25:1 | about 5:1 |
| Humulene | About 1:100 to | About 1:25 to | About 1:5 to |
| | about 100:1 | about 25:1 | about 5:1 |
| Linalool | About 1:100 to | About 1:25 to | About 1:5 to |
| | about 100:1 | about 25:1 | about 5:1 |
| Adrenaline | About 1:100 to | About 1:25 to | About 1:5 to |
| | about 100:1 | about 25:1 | about 5:1 |
| Amineptine | About 1:100 to | About 1:25 to | About 1:5 to |
| | about 100:1 | about 25:1 | about 5:1 |
| Erinacine A | About 1:100 to | About 1:25 to | About 1:5 to |
| | about 100:1 | about 25:1 | about 5:1 |
| Hericenone A | About 1:100 to | About 1:25 to | About 1:5 to |
| | about 100:1 | about 25:1 | about 5:1 |
| Phenelzine | About 1:100 to | About 1:25 to | About 1:5 to |
| | about 100:1 | about 25:1 | about 5:1 |

Exemplary pharmaceutical compositions of an alkyl quaternary tryptamine compound of the disclosure and a second compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone and an excipient with exemplary molar ratios of an alkyl quaternary tryptamine compound to the second compound are shown in Table 2. An alkyl quaternary tryptamine compound of the disclosure may be any one of the exemplary embodiments described above including their crystalline forms as disclosed herein.

TABLE 2

| | Molar ratio of an | Molar ratio of an | Molar ratio of an |
|---|---|---|---|
| | alkyl quaternary | alkyl quaternary | alkyl quaternary |
| | tryptamine | tryptamine | tryptamine |
| | compound:second | compound:second | compound:second |
| Second Compound | compound | compound | compound |
| 3,4- | About 1:100 to | About 1:25 to | About 1:5 to |
| methylenedioxymethamphetamine | about 100:1 | about 25:1 | about 5:1 |
| Citalopram | About 1:100 to | About 1:25 to | About 1:5 to |
| | about 100:1 | about 25:1 | about 5:1 |
| Escitalopram | About 1:100 to | About 1:25 to | About 1:5 to |
| | about 100:1 | about 25:1 | about 5:1 |
| Fluoxetine | About 1:100 to | About 1:25 to | About 1:5 to |
| | about 100:1 | about 25:1 | about 5:1 |
| Paroxetine | About 1:100 to | About 1:25 to | About 1:5 to |
| | about 100:1 | about 25:1 | about 5:1 |
| Sertraline | About 1:100 to | About 1:25 to | About 1:5 to |
| | about 100:1 | about 25:1 | about 5:1 |
| [3-(2-Dimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxytryptamine | About 1:100 to | About 1:25 to | About 1:5 to |
| | about 100:1 | about 25:1 | about 5:1 |
| 4-hydroxy-N,N-dimethyltryptamine | About 1:100 to | About 1:25 to | About 1:5 to |
| | about 100:1 | about 25:1 | about 5:1 |
| [3-(2-methylaminoethyl)-1H-indol- | About 1:100 to | About 1:25 to | About 1:5 to |
| 4-yl] dihydrogen phosphate | about 100:1 | about 25:1 | about 5:1 |
| 4-hydroxy-N-methyltryptamine | About 1:100 to | About 1:25 to | About 1:5 to |
| | about 100:1 | about 25:1 | about 5:1 |
| [3-(aminoethyl)-1H-indol-4-yl] | About 1:100 to | About 1:25 to | About 1:5 to |
| dihydrogen phosphate | about 100:1 | about 25:1 | about 5:1 |
| [3-(2-trimethylaminoethyl)-1H- | About 1:100 to | About 1:25 to | About 1:5 to |
| indol-4-yl] dihydrogen phosphate | about 100:1 | about 25:1 | about 5:1 |
| 4-hydroxy-N,N,N- | About 1:100 to | About 1:25 to | About 1:5 to |
| trimethyltryptamine | about 100:1 | about 25:1 | about 5:1 |
| THC | About 1:100 to | About 1:25 to | About 1:5 to |
| | about 100:1 | about 25:1 | about 5:1 |
| CBC | About 1:100 to | About 1:25 to | About 1:5 to |
| | about 100:1 | about 25:1 | about 5:1 |
| CBD | About 1:100 to | About 1:25 to | About 1:5 to |
| | about 100:1 | about 25:1 | about 5:1 |
| CBG | About 1:100 to | About 1:25 to | About 1:5 to |
| | about 100:1 | about 25:1 | about 5:1 |
| Myrcene | About 1:100 to | About 1:25 to | About 1:5 to |
| | about 100:1 | about 25:1 | about 5:1 |
| Pinene | About 1:100 to | About 1:25 to | About 1:5 to |
| | about 100:1 | about 25:1 | about 5:1 |
| Caryophyllene | About 1:100 to | About 1:25 to | About 1:5 to |
| | about 100:1 | about 25:1 | about 5:1 |
| Limonene | About 1:100 to | About 1:25 to | About 1:5 to |
| | about 100:1 | about 25:1 | about 5:1 |
| Humulene | About 1:100 to | About 1:25 to | About 1:5 to |
| | about 100:1 | about 25:1 | about 5:1 |
| Linalool | About 1:100 to | About 1:25 to | About 1:5 to |
| | about 100:1 | about 25:1 | about 5:1 |
| Adrenaline | About 1:100 to | About 1:25 to | About 1:5 to |
| | about 100:1 | about 25:1 | about 5:1 |
| Amineptine | About 1:100 to | About 1:25 to | About 1:5 to |
| | about 100:1 | about 25:1 | about 5:1 |
| Erinacine A | About 1:100 to | About 1:25 to | About 1:5 to |
| | about 100:1 | about 25:1 | about 5:1 |
| Hericenone A | About 1:100 to | About 1:25 to | About 1:5 to |
| | about 100:1 | about 25:1 | about 5:1 |
| Phenelzine | About 1:100 to | About 1:25 to | About 1:5 to |
| | about 100:1 | about 25:1 | about 5:1 |

An “effective amount” or a “therapeutically effective amount” of an alkyl quaternary tryptamine compound of the disclosure is generally in the range of about 0.1 to about 100 mg daily (oral dose), of about 0.1 to about 50 mg daily (oral dose) of about 0.25 to about 25 mg daily (oral dose), of about 0.1 to about 5 mg daily (oral dose) or of about 0.5 to about 2.5 mg daily (oral dose). The actual amount required for treatment of any particular patient may depend upon a variety of factors including, for example, the disease being treated and its severity; the specific pharmaceutical composition employed; the age, body weight, general health, sex, and diet of the patient; the mode of administration; the time of administration; the route of administration; and the rate of excretion; the duration of the treatment; any drugs used in combination or coincidental with the specific compound employed; and other such factors well known in the medical arts. These factors are discussed in Goodman and Gilman's “The Pharmacological Basis of Therapeutics,” Tenth Edition, A. Gilman, J. Hardman and L. Limbird, eds., McGraw-Hill Press, 155-173 (2001), which is incorporated herein by reference. An alkyl quaternary tryptamine compound of the disclosure and pharmaceutical compositions containing it may be used in combination with other agents that are generally administered to a patient being treated for psychological and other disorders discussed above. They may also be co-formulated with one or more of such agents in a single pharmaceutical composition.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. Exemplary carriers include those that do not substantially alter the structure or activity of alkyl quaternary tryptamine compound of the disclosure, nor produce undesirable biological effects or otherwise interact in a deleterious manner with any other component(s) of the pharmaceutical composition.

The pharmaceutical compositions of the disclosure may be prepared by methods know in the pharmaceutical formulation art, for example, see Remington's Pharmaceutical Sciences, 18th Ed., (Mack Publishing Company, Easton, Pa., 1990), which is incorporated herein by reference. In a solid dosage form, a 4-HO-DPT compound of the disclosure may be admixed with at least one pharmaceutically acceptable excipient such as, for example, sodium citrate or dicalcium phosphate or (a) fillers or extenders, such as, for example, starches, lactose, sucrose, glucose, mannitol, and silicic acid, (b) binders, such as, for example, cellulose derivatives, starch, alignates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like, (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents. In some embodiments, the excipient is not water. In some embodiments, the excipient is not a solvent (e.g., EtOH, diethyl ether, ethyl acetate, or hydrocarbon-based solvents (e.g., hexanes). In some embodiments, the dosage form is substantially free of water and/or solvents, for example less than about 5% water by mass, less than 2% water by mass, less than 1% water by mass, less than 0.5% water by mass, or less than 0.1% water by mass.

Excipients or pharmaceutically acceptable adjuvants known in the pharmaceutical formulation art may also be used in the pharmaceutical compositions of the disclosure. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a pharmaceutical composition of the disclosure may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms as described above may be prepared with coatings and shells, such as enteric coatings and others well known in the art. They may contain pacifying agents and can also be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Solid dosage forms for oral administration, which includes capsules, tablets, pills, powders, and granules, may be used. In such solid dosage forms, the active compound may be mixed with at least one inert, pharmaceutically acceptable excipient (also known as a pharmaceutically acceptable carrier).

Administration of alkyl quaternary tryptamine compounds of the disclosure in pure form or in an appropriate pharmaceutical composition may be carried out via any of the accepted modes of administration or agents for serving similar utilities. Thus, administration may be, for example, orally, buccally, nasally, parenterally (intravenous, intramuscular, or subcutaneous), topically, transdermally, intravaginally, intravesically, or intrasystemically, in the form of solid, semi-solid, lyophilized powder, or liquid dosage forms, such as, for example, tablets, suppositories, pills, soft elastic and hard gelatin capsules, powders, solutions, suspensions, or aerosols, or the like, such as, for example, in unit dosage forms suitable for simple administration of precise dosages. One route of administration may be oral administration, using a convenient daily dosage regimen that can be adjusted according to the degree of severity of the disease-state to be treated.

### EXAMPLES

### Example 1: 4-Acetoxy-N,N,N-trimethyltryptammonium Iodide (4-AcO-TMT Iodide)

4-acetoxy-N,N-dimethyltryptammine (4-AcO-DMT) fumarate was stirred in a 1:1 solution of methanol and iodomethane and heated to reflux for four hours. Solvent was removed in vacuo, and a white powder is obtained after triturating and washing the resulting residue with tetrahydrofuran. ¹H NMR (400 MHz, D₂O): δ 7.46 (d, J=8.1 Hz, 1H, ArH), 7.33 (s, 1H, ArH), 7.27 (t, J=8.0 Hz, 1H, ArH), 6.90 (d, J=7.8 Hz, 1H, ArH), 3.63 (t, J=7.5 Hz, 2H, CH₂), 3.27 (t, J=8.6 Hz, 2H, CH₂), 3.21 (s, 9H, NCH₃), 2.47 (s, 3H, C(O)CH₃). Unit Cell: a=7.8459 (9) Å, b=9.8098 (12) Å, c=11.0823 (12) Å, α=90°, β=101.069 (3)°, γ=90°, V=837.10 (17) Å³.

Second Preparation of 4-AcO-TMT iodide: 250 mg of 4-acetoxy-N,N-dimethyltryptammonium (4-AcO-DMT) fumarate was dissolved in 10 mL of methanol in a 50 mL round bottom flask, and 10 mL of iodomethane was then added. The mixture was stirred for 24 hours under an atmosphere of dinitrogen. The solvent was removed in vacuo. The resulting powder was washed with diethyl ether and filtered to yield 313 mg of yellow powder. This powder was dissolved in 75 mL of acetone. The solution was heated with stirring and reduced in volume to 40 mL. The mixture was cooled in an ice bath, yielding a white precipitate. The powder was filtered to yield 142 mg of white powder (53.01% yield). ¹H NMR (400 MHz, D₂O): δ 7.46 (d, J=8.6 Hz, 1H, ArH), 7.34 (s, 1H, ArH), 7.22 (t, J=7.8 Hz, 1H, ArH), 6.95 (d, J=8.3 Hz, 1H, ArH) 3.63 (t, J=7.9 Hz, 2H, CH₂), 3.28 (t, J=8.0 Hz, 2H, CH₂), 3.21 (s, 9H, CH₃), 2.47 (s, 3H, C(O)CH₃); ¹³C NMR (100 MHz, D₂O): δ 174.32 (C═O), 143.59 (ArC), 139.28 (ArC), 125.82 (ArC), 123.01 (ArC), 119.11 (ArC), 112.92 (ArC), 111.13 (ArC), 107.32 (ArC), 67.64 (NCH₂), 53.73 (NCH₃), 21.25 (CH₂), 20.48 (C(O)CH₃). Elemental analysis calcd. for C₁₅H₂₁N₂O₂I: C, 46.40, H, 5.45, N, 7.22; Found: C, 46.17, H, 5.35, N, 7.11.

X-Ray data collection and refinement details for 4-AcO-TMT iodide: Crystals suitable for X-ray diffraction studies were grown from the slow evaporation of an aqueous solution. All operations were performed on a Bruker D8 Venture CMOS diffractometer, using Mo Kα radiation with a TRIUMPH monochromator at a temperature of 298 K. Data collection was carried out using the Bruker APEX3 software. Cell refinement and data reduction were performed with the SAINT program. The structure solution was done with SHELXS and structure refinement was performed with SHELXL. Further refinement and molecular graphics were generated using the OLEX2 and Mercury CSD software.

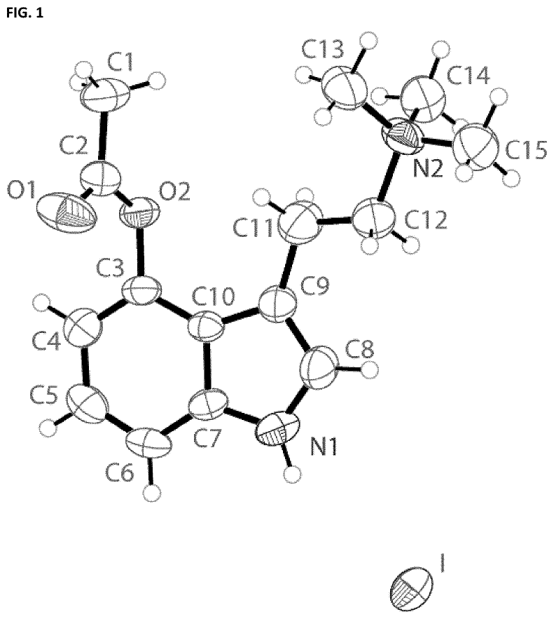

All non-hydrogen atoms were refined anisotropically (XL) by full matrix least squares on F². Hydrogen atom H1 was found from a Fourier difference map, and refined with a fixed distance of 0.87 Å. Isotropic displacement parameters were set to 1.20 times U_{eq} of the parent N atoms. The remaining hydrogen atoms were placed in calculated positions and then refined with a riding model with C—H lengths of 0.93 Å (sp²), 0.96 Å (CH₃) and 0.97 Å (CH₂) with isotropic displacement parameters set to 1.20 (sp² and CH₂) and 1.50 (CH₃) times U_{eq} of the parent C atom. Further details are in Table 3. FIG. 1 is the fully labelled displacement ellipsoid representation (50%) of the asymmetric unit of 4-AcO-TMT iodide.

TABLE 3
Crystal data and structure refinement for 4-AcO-TMT iodide.

| Empirical formula | C₁₅H₂₁IN₂O₂ |
|---|---|
| Formula weight | 388.24 |
| Temperature/K | 299.05 |
| Crystal system | Monoclinic |
| Space group | P2₁ |
| a/Å | 7.8459(9) |
| b/Å | 9.8098(12) |
| c/Å | 11.0823(12) |
| α/° | 90 |
| β/° | 101.069(3) |
| γ/° | 90 |
| Volume/Å³ | 837.10(17) |
| Z | 2 |
| ρ_{calc}g/cm³ | 1.540 |
| μ/mm⁻¹ | 1.916 |
| F(000) | 388.0 |
| Crystal size/mm³ | 0.2 × 0.1 × 0.05 |
| Radiation | MoKα (λ = 0.71073) |
| 2Θ range for data collection/° | 5.866 to 50.852 |
| Index ranges | −9 ≤ h ≤ 9, −11 ≤ k ≤ |
| | 11, −13 ≤ l ≤ 13 |
| Reflections collected | 18328 |
| Independent reflections | 3076 [Rᵢₙₜ = 0.0424, R_{sigma} = 0.0298] |
| Data/restraints/parameters | 3076/2/189 |
| Goodness-of-fit on F² | 1.064 |
| Final R indexes [I ≥ 2σ (I)] | R₁ = 0.0300, wR₂ = 0.0548 |
| Final R indexes [all data] | R₁ = 0.0396, wR₂ = 0.0583 |
| Largest diff. peak/hole/e Å⁻³ | 0.51/−0.40 |
| Flack parameter | −0.001(11) |

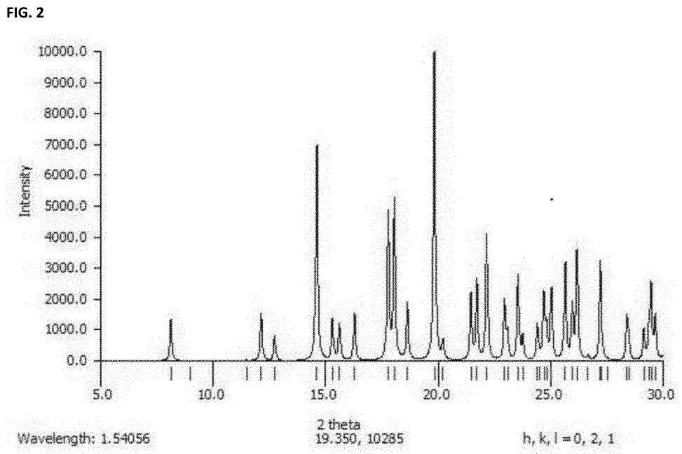

FIG. 2 is a simulated x-ray powder diffraction (XRPD) pattern of crystalline 4-Acetoxy-N,N,N-trimethyltryptammonium Iodide (4-AcO-TMT iodide) from its single crystal data. Crystalline 4-Acetoxy-N,N,N-trimethyltryptammonium Iodide (4-AcO-TMT iodide) may be characterized by the XRPD peaks at 8.1, 14.6 and 19.8° 2θ±0.2°2θ as well as by an XRPD pattern substantially similar to FIG. 2.

### Example 2: 4-Hydroxy-N,N,N-trimethyltryptammonium (4-HO-TMT) Iodide

4-acetoxy-N,N,N-trimethyltryptammonium (4-AcO-TMT) iodide was stirred in a 1:1 solution of water and acetic acid in air for 12 hours. Solvent was removed in vacuo, and a white powder is obtained after triturating and washing the resulting residue with tetrahydrofuran. ¹H NMR (400 MHz, D₂O): δ 7.18 (s, 1H, ArH), 7.12-7.06 (m, 2H, ArH), 6.57 (dd, J=6.0, 2.4 Hz, 1H, ArH), 3.62 (t, J=7.8 Hz, 2H, CH₂), 3.37 (t, J=8.1 Hz 2H, CH₂), 3.19 (s, 9H, NCH₃). Unit Cell: a=11.3057 (9) Å, b=11.2370 (10) Å, c=12.7785 (10) A, α=90°, β=113.087 (2)°, γ=900, V=1493.4 (2) Å³.

Second Preparation of 4-HO-TMT Iodide

95 mg of 4-AcO TMT iodide was dissolved in 4 mL of deionized (DI) water in a 50 mL round bottom flask. 20 mL of acetic acid was added to the mixture, and it was refluxed under an atmosphere of dinitrogen for 2 days. Solvent was removed in vacuo to obtain a green/blue oil. 3 mL of methanol and 20 mL of ethyl acetate were added to the green/blue oil, leaving a green/blue powder that was removed via filtration. Solvent was removed in vacuo. The resulting oil was dissolved n 5 mL of ethanol, and 30 mL of pentane was added to generate a precipitate. The resulting powder was isolated via filtration to give 53.1 g (60% yield) to give an off-white powder. ¹H NMR (400 MHz, D₂O): δ 7.19 (s, 1H, ArH), 7.12-7.07 (m, 2H, ArH), 6.56 (dd, J=5.9, 2.5 Hz, 1H, ArH), 3.66-3.62 (m, 2H, CH₂), 3.40-3.36 (m, 2H, CH₂), 3.20 (s, 9H, CH₃); ¹³C NMR (100 MHz, D₂O): δ 150.62 (ArC), 139.37 (ArC), 123.89 (ArC), 123.86 (ArC), 116.56 (ArC), 108.72 (ArC), 105.23 (ArC), 104.43 (ArC), 68.20 (NCH₂), 53.59 (NCH₃), 21.20 (CH₂). Elemental analysis calcd. For C₁₃H₁₉N₂OI: C, 45.10, H, 5.53, N, 8.09; Found: C, 44.84, H, 5.23, N, 7.98.

X-Ray Data Collection and Refinement Details for 4-HO-TMT Iodide

Crystals suitable for X-ray diffraction studies were grown from the slow evaporation of an aqueous solution. All operations were performed on a Bruker D8 Venture CMOS diffractometer, using Mo Kα radiation with a TRIUMPH monochromator at a temperature of 200 K. Data collection was carried out using the Bruker APEX3 software. Cell refinement and data reduction were performed with the SANT program. The structure solution was done with SHELXS and structure refinement was performed with SHEXL. Further refinement and molecular graphics were generated using the OLEX2 and Mercury CSD software.

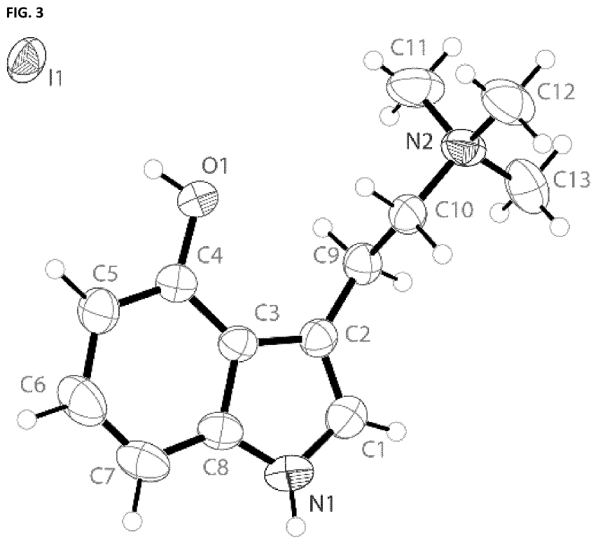

All non-hydrogen atoms were refined anisotropically (XL) by full matrix least squares on F². Hydrogen atom H1 and H1 Å were found from a Fourier difference map, and refined with a fixed distance of 0.86 Å and 0.85 Å respectively. Isotropic displacement parameters were set to 1.20 times U_{eq} of the parent N atom, and 1.50 times U_{eq} of the parent 0 atom. The remaining hydrogen atoms were placed in calculated positions and then refined with a riding model with C—H lengths of 0.95 Å (sp²), 0.98 Å (CH₂) and 0.99 Å (CH₃) with isotropic displacement parameters set to 1.20 (sp² and CH₂) and 1.50 (CH₃) times U_{eq} of the parent C atom. Further details are in Table 4. FIG. 3 is the fully labelled displacement ellipsoid representation (50%) of the asymmetric unit of 4-HO-TMTI.

TABLE 4
Crystal data and structure refinement for 4-HO-TMT iodide.

| Empirical formula | C₁₃H₁₉IN₂O |
|---|---|
| Formula weight | 346.20 |
| Temperature/K | 200.0 |
| Crystal system | Monoclinic |
| Space group | P2₁/n |
| a/Å | 11.3057(9) |
| b/Å | 11.2370(10) |
| c/Å | 12.7785(10) |
| α/° | 90 |
| β/° | 113.087(2) |
| γ/° | 90 |
| Volume/Å³ | 1493.4(2) |
| Z | 4 |
| ρ_{calc}g/cm³ | 1.540 |
| μ/mm⁻¹ | 2.133 |
| F(000) | 688.0 |
| Crystal size/mm³ | 0.4 × 0.2 × 0.1 |
| Radiation | MoKα (λ = 0.71073) |
| 2Θ range for data collection/° | 6.164 to 52.884 |
| Index ranges | −13 ≤ h ≤ 14, −14 ≤ k ≤ |
| | 14, −15 ≤ l ≤ 15 |
| Reflections collected | 29269 |
| Independent reflections | 3063 [Rᵢₙₜ = 0.0564, R_{sigma} = 0.0277] |
| Data/restraints/parameters | 3063/2/161 |
| Goodness-of-fit on F² | 1.073 |
| Final R indexes [I ≥ 2σ (I)] | R₁ = 0.0368, wR₂ = 0.0797 |
| Final R indexes [all data] | R₁ = 0.0574, wR₂ = 0.0885 |
| Largest diff. peak/hole/e Å⁻³ | 1.05/−0.93 |

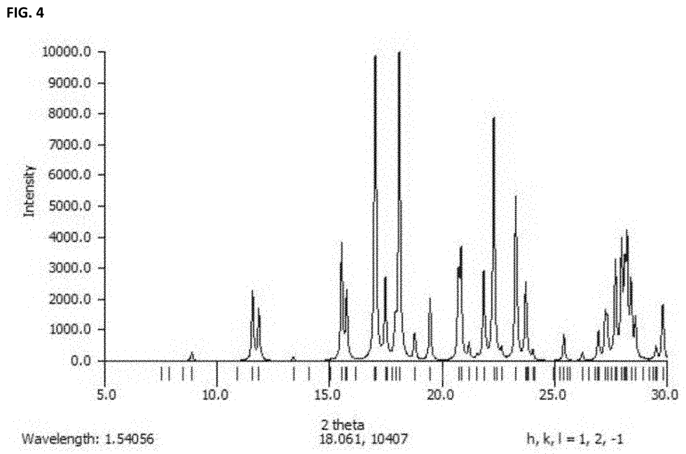

FIG. 4 is a simulated x-ray powder diffraction (XRPD) pattern of crystalline 4-Hydroxy-N,N,N-trimethyltryptammonium Iodide (4-HO-TMT iodide) from its single crystal data. Crystalline 4-Hydroxy-N,N,N-trimethyltryptammonium Iodide (4-HO-TMT iodide) may be characterized by the XRPD peaks at 17.0, 18.1 and 19.5° 2θ±0.2°2θ as well as by an XRPD pattern substantially similar to FIG. 4.

### Example 3: Cellular Assays

Cellular assays were performed by Eurofins CEREP SA, Celle-Levescault, France. All receptors were separately expressed in HEK-293 cells. Cell membrane homogenates (30 μg protein) are incubated for 60 min (5-HT_{1A}, 5-HT_{2A}, 5-HT₂B) or 120 min (5-HT₃) at 22° C. with radiolabeled ligand in the absence or presence of the test compound in a buffer containing 50 mM Tris-HCl (pH 7.4), 5 mM MgCl₂, 10 μM pargyline and 0.1% ascorbic acid. For 5-HT3, the buffer contained 50 mM Tris-HCl (pH 7.4), 5 mM MgCl₂, and 1 mM EDTA. Binding was reported as the Kᵢ for the inhibition of binding of well-characterized orthosteric ligands. The ligands used for each receptor were:

5-HT_{1A}: [³H] 8-OH-DPAT

5-HT2 Å: [¹²⁵I] (±)DOI

5-HT2B: [¹²⁵I] (±)DOI

5-HT₃: [³H] BRL 43694

Nonspecific binding was determined in the presence of 1 μM unlabeled ligand listed above. Following incubation, the samples were filtered rapidly under vacuum through glass fiber filters (GF/B, Packard) presoaked with 0.3% PEI and rinsed several times with ice-cold 50 mM Tris-HCl using a 96-sample cell harvester (Unifilter, Packard). The filters were dried then counted for radioactivity in a scintillation counter (Topcount, Packard) using a scintillation cocktail (Microscint 0, Packard). The results are expressed as a percent inhibition of the control radioligand specific binding.

The IC₅₀ values and Hill coefficients (nH) were determined by non-linear regression analysis of the competition curves generated with mean replicate values using Hill equation curve fitting Y=D+[(A−D)/(1+(C/C₅₀)ⁿH)] where Y=specific binding, A=left asymptote of the curve, D=right asymptote of the curve, C=compound concentration, C₅₀=IC₅₀, and nH=slope factor.

Analysis was performed using software developed at Cerep (Hill software) and validated by comparison with data generated by the commercial software SigmaPlot® 4.0 for Windows® (© 1997 by SPSS Inc.). The inhibition constants (Kᵢ) were calculated using the Cheng Prusoff equation: Kᵢ=IC₅₀ (1+L/K_{D}), where L=concentration of radioligand in the assay, and K_{D}=affinity of the radioligand for the receptor. A scatchard plot was used to determine the K_{D}.

The results in terms of inhibition constants (Kᵢ) are shown in Table 5. The aeruginascin active metabolite, 4-HO-TMTI, shows activity at 5-HT_{1A}, 5-HT_{2A} and 5-HT_{2B}. Counter to the prevailing theory that aeruginascin should function as a powerful 5-HT₃ agonist, there is no activity observed at this receptor. The aeruginascin functional analogue, 4-AcO-TMTI shows no activity at any of the receptors. For comparison, psilocybin, the pro-drug of psilocin, shows no activity at 5-HT_{1A}, 5-HT_{2A}, nor 5-HT₃, but does show itself to be a potent 5-HT_{2B} agonist. Psilocin, its active metabolite, shows activity at 5-HT_{1A} and 5-HT_{2A} that is more active though comparable to 4-HO-TMTI. (Roth et al.) It is significantly more potent than 4-HO-TMTI at the 5-HT_{2B} receptor, and in fact, psilocybin is more active at this receptor as well.

TABLE 5
Inhibition constants (Kᵢ) in nM units.

| Compound | 5-HT_{1A} | 5-HT_{2A} | 5-HT_{2B} | 5-HT₃ |
|---|---|---|---|---|
| 4-HO-TMTI | 4,400 | 670 | 120 | >10,000 |
| 4-AcO-TMTI | >10,000 | >10,000 | >10,000 | >10,000 |
| Psilocin^{‡} | 567.4 | 107.2 | 4.6 | >10,000 |
| Psilocybin^{‡} | >10,000 | >10,000 | 98.7 | >10,000 |

### Example 4: N,N-di-methyl-N-propyl-tryptammonium (DMPT) Iodide

N,N-di-methyl-N-propyl-tryptammonium (DMPT) iodide was prepared by mixing 101 mg of a commercial sample of N-methyl-N-propyl-tryptamine (The Indole Shop) and 4 mL of methyl iodide in 4 mL of methanol. The mixture was refluxed for twelve hours under an atmosphere of nitro-gen. The solvent was removed in vacuo, and the remaining residue was recrystallized from ethanol to yield colourless single crystals suitable for X-ray diffraction studies. The product was also characterized by nuclear magnetic resonance. ¹H NMR (400 MHz, D₂O): d 7.69 (d, J=8.0 Hz, 1H, ArH), 7.55 (d, J=8.2 Hz, 1H, ArH), 7.33-7.28 (m, 2H, ArH), 7.22 (t, J=7.0 Hz, 1H, ArH), 3.60 (m, 2H, CH₂), 3.36 (m, 4H, CH₂), 3.17 (s, 6H, CH₃), 1.82 (m, 2H, CH₂), 0.97 (t, J=7.0 Hz, 3H, CH₃).

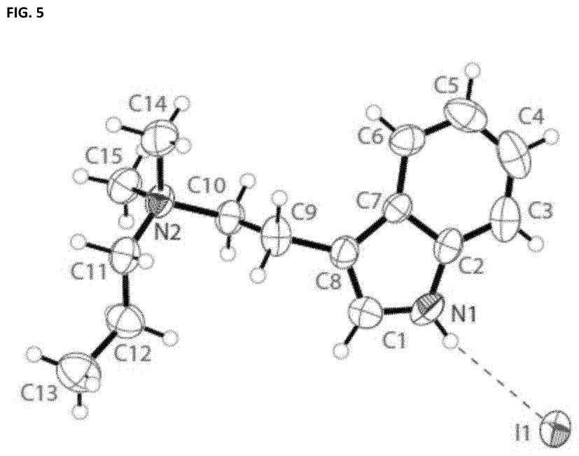

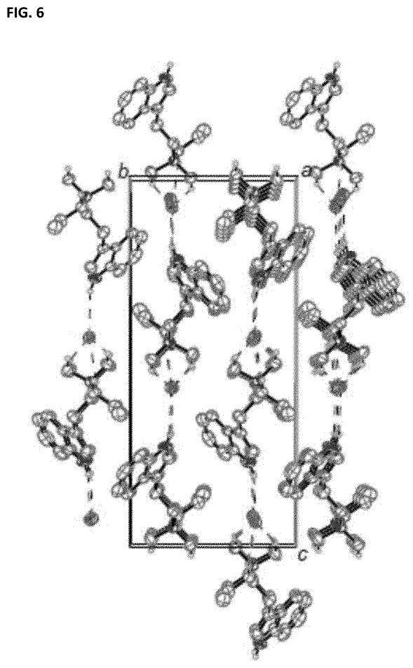

The molecular structure of crystalline DMPT iodide is shown in FIG. 5. Crystal data, data collection and structure refinement details are summarized in Table 6. The asymmetric unit contains one N,N-di-methyl-N-n-propyl tryptammonium (C₁₅H₂₃N₂⁺) cation and one iodide anion. The indole ring of the cation is near, planar with a mean deviation from planarity of 0.011 Å. The ethyl-ammonium arm is turned away from the plane with a C7-C8-C9-C10 torsion angle of 89.1 (4)°. The DMPT cation and the iodide anion are held together in the asymmetric unit via N(1)−H(1) . . . I(1) hydrogen bonds, between the indole nitrogen and the iodide. The packing of crystalline DMPT iodide is shown in FIG. 6.

Crystal data, data collection and structure refinement details are summarized in Table 6.

TABLE 6
Crystalline DMPT Iodide

| | |
|---|---|
| Chemical formula | I•C₁₅H₂₃N₂ |
| Mᵣ | 358.25 |
| Crystal system, space group | Monoclinic, P2₁/c |
| Temperature (K) | 303 |
| a, b, c (Å) | 7.4471 (6), 9.9016 (9), 22.052 (2) |
| β (°) | 94.184 (3) |
| V (Å³) | 1621.8 (2) |
| Z | 4 |
| Radiation type | Mo Ka |
| m (mm⁻¹) | 1.96 |
| Crystal size (mm) | 0.40 × 0.14 × 0.12 |
| Diffractometer | Bruker D8 Venture CMOS |
| Absorption correction | Multi-scan |
| | SADABS2016/2 (Bruker, 2016/2) was used for absorption |
| | correction. wR2(int) was 0.0600 before and 0.0507 after |
| | correction. The Ratio of minimum to maximum transmission is |
| | 0.8362. The I/2 correction factor is Not present. |
| Tₘᵢₙ, Tₘₐₓ | 0.470, 0.562 |
| No. of measured, independent and | 44530, 3071, 2362 |
| observed [I > 2s(I)] reflections | |
| Rᵢₙₜ | 0.036 |
| (sin q/I)ₘₐₓ (Å⁻¹) | 0.611 |
| R[F² > 2s(F²)], wR(F²), S | 0.028, 0.054, 1.13 |
| No. of reflections | 3071 |
| No. of parameters | 170 |
| No. of restraints | 1 |
| H-atom treatment | H atoms treated by a mixture of independent and constrained |
| | refinement |
| Dρₘₐₓ, Dρₘᵢₙ (e Å⁻³) | 0.53, −0.47 |
| Absolute structure | — |
| Absolute structure parameter | — |

Computer programs: APEX3 (Bruker, 2018), SAINT (Bruker, 2018), SHELXT2014 (Sheldrick, 2015a), SHELXL2018 (Sheldrick, 2015b), Olex2 (Dolomanov et al., 2009), pubICIF (Westrip, 2010).

TABLE 7
Hydrogen-bond geometry (Å, °) for Crystalline DMPT Iodide

| D-H . . . A | D-H | H . . . A | D . . . A | D-H . . . A |
|---|---|---|---|---|
| N1-H1 . . . I1 | 0.86 (1) | 2.91 (2) | 3.733 (3) | 162 (3) |

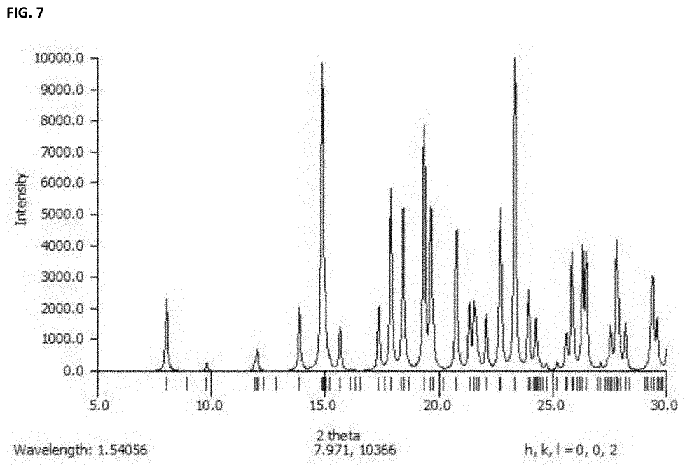

FIG. 7 is a simulated x-ray powder diffraction (XRPD) pattern of crystalline N,N-dimethyl-N-propyl (DMPT) iodide from its single crystal data. Crystalline N,N-dimethyl-N-propyl (DMPT) iodide may be characterized by the XRPD peaks at 8.0, 17.9 and 23.3°2θ±0.2°2θ as well as by an XRPD pattern substantially similar to FIG. 7.

### Example 5: N,N-di-methyl-N-allyl-tryptammonium (DMALT) Iodide

N,N-di-methyl-N-allyl-tryptammonium (DMALT) iodide was prepared by mixing 101 mg of a commercial sample of N-allyl-N-methyl-tryptamine (The Indole Shop) with 4 mL of methyl iodide in 4 mL of methanol. The mixture was refluxed for twelve hours under an atmosphere of nitrogen. The solvent was removed in vacuo, and the remaining residue was recrystallized from acetone to yield colourless crystals suitable for X-ray diffraction studies. The product was also characterized by nuclear magnetic resonance. ¹H NMR (400 MHz, D₂O): d 7.69 (d, J=7.8 Hz, 1H, ArH), 7.55 (d, J=8.2 Hz, 1H, ArH), 7.32-7.28 (m, 1H, ArH), 7.22 (t, J=7.2 Hz, 1H, ArH), 6.13-6.03 (m, 1H, CH), 5.77-5.71 (m, 2H, CH₂), 4.04 (d, J=7.3 Hz, 1H, CH₂), 3.61-3.56 (m, 2H, CH₂), 3.37-3.32 (m, 2H, CH₂), 3.17 (s, 6H, CH₃).

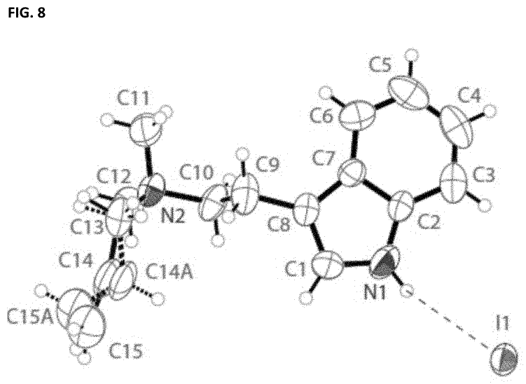

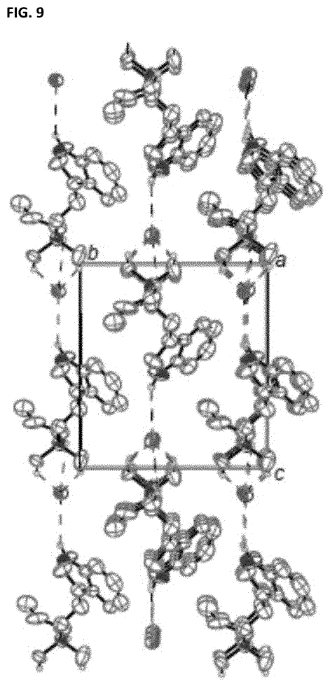

The molecular structure of crystalline DMALT iodide is shown in FIG. 8. The asymmetric unit contains one N-allyl-N,N-di-methyl-tryptammonium (C₁₅H₂₁N₂⁺) cation and one iodide anion. The indole ring of the cation is near planar, with a mean deviation from planarity of 0.013 Å. The ethyl-ammonium arm is turned away from the plane with a C7-C8-C9-C10 torsion angle of 101.9 (9)°. The allyl group is disordered over two orientations with a 0.30 (4) to 0.70 (4) occupancy ratio for C14, C15 and C14 Å, C15 Å, respectively. The DMALT structure is very similar to that of DMPT, with the ions held together in the asymmetric unit through N(1)−H(1) . . . I(1) hydrogen bonds. The packing of crystalline DMALT iodide is shown in FIG. 9.

Crystal data, data collection and structure refinement details are summarized in Table 8.

TABLE 8
Crystalline DMALT Iodide

| Chemical formula | 0.5(I)•0.5(C₁₅H₂₁N₂) |
|---|---|
| Mᵣ | 178.12 |
| Crystal system, space group | Monoclinic, P2₁ |
| Temperature (K) | 303 |
| a, b, c (Å) | 7.3471 (8), 9.9672 (9), 10.9499 (11) |
| β (°) | 94.671 (3) |
| V (Å³) | 799.20 (14) |
| Z | 4 |
| Radiation type | Mo Ka |
| m (mm⁻¹) | 1.99 |
| Crystal size (mm) | 0.39 × 0.22 × 0.15 |
| Diffractometer | Bruker D8 Venture CMOS |
| Absorption correction | Multi-scan SADABS2016/2 (Bruker, 2016/2) was used for |
| | absorption correction. wR2(int) was 0.0671 before and 0.0484 |
| | after correction. The Ratio of minimum to maximum |
| | transmission is 0.8154. The I/2 correction factor is Not present. |
| Tₘᵢₙ, Tₘₐₓ | 0.608, 0.745 |
| No. of measured, independent and | 26314, 3038, 2868 |
| observed [I > 2s(I)] reflections | |
| Rᵢₙₜ | 0.031 |
| (sin q/I)ₘₐₓ (Å⁻¹) | 0.611 |
| R[F² > 2s(F²)], wR(F²), S | 0.027, 0.071, 1.13 |
| No. of reflections | 3038 |
| No. of parameters | 174 |
| No. of restraints | 5 |
| H-atom treatment | H-atom parameters constrained |
| Dρₘₐₓ, Dρₘᵢₙ (e Å⁻³) | 0.46, −0.48 |
| Absolute structure | Refined as an inversion twin. |
| Absolute structure parameter | 0.29 (5) |

Computer programs: APEX3 (Bruker, 2018), SAINT (Bruker, 2018), SHELXT2014 (Sheldrick, 2015a), SHELXL2018 (Sheldrick, 2015b), Olex2 (Dolomanov et al., 2009), pubICIF (Westrip, 2010).

TABLE 9
Hydrogen-bond geometry (Å, °) for Crystalline DMALT Iodide

| D-H . . . A | D-H | H . . . A | D . . . A | D-H . . . A |
|---|---|---|---|---|
| N1-H1 . . . I1 | 0.86 | 2.95 | 3.727 (6) | 152 |

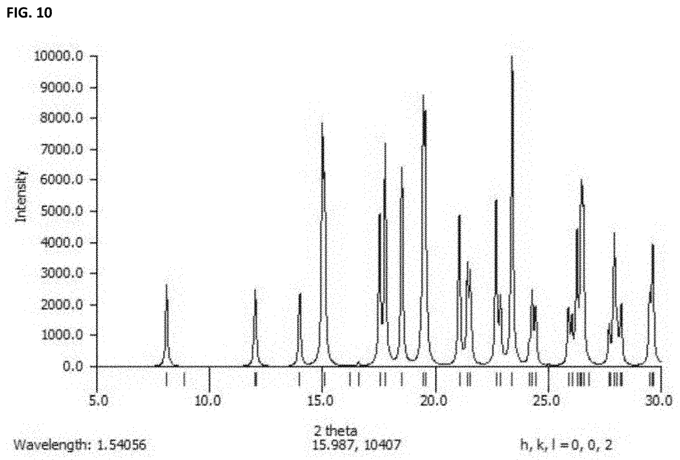

FIG. 10 is a simulated x-ray powder diffraction (XRPD) pattern of crystalline N,N-dimethyl-N-allyl (DMALT) iodide from its single crystal data. Crystalline N,N-dimethyl-N-allyl (DMALT) iodide may be characterized by the XRPD peaks at 12.0, 18.5 and 23.4° 2θ±0.2°2θ as well as by an XRPD pattern substantially similar to FIG. 10.

### Example 6: 4-acetoxy-N,N-dimethyl-N-ethyltryptammonium (4-AcO-DMET) Iodide

Synthesis: 300 mg of 4-acetoxy-N,N-dimethyl-tryptammonium (4-AcO-DMT) fumarate was dissolved in 30 mL of tetrahydrofuran, and 6 mL of iodoethane was added. The mixture was refluxed overnight under an atmosphere of nitrogen. This resulted in the precipitation of a white powder from the yellow solution. The precipitate was isolated via vacuum filtration to give white powder, which was washed with diethyl ether to yield 303 mg of pure product (91% yield). ¹H NMR (400 MHz, D₂O): δ 7.46 (dd, J=8.2, 0.7 Hz, 1H, ArH), 7.33 (s, 1H, ArH), 7.25 (t, J=7.9 Hz, 1H, ArH), 6.90 (dd, J=7.7, 0.7 Hz, 1H, ArH), 3.58-3.54 (m, 2H, CH₂), 3.46 (q, J=7.3 Hz, 2H, CH₂), 3.25-3.20 (m, 2H, CH₂), 3.12 (s, 6H, CH₃), 2.47 (s, 3H, (CO)CH₃), 1.38-1.35 (m, 3H, CH₃); ¹³C NMR (100 MHz, D₂O): δ 174.3 (CO), 143.6 (ArC), 139.2 (ArC), 125.7 (ArC), 123.0 (ArC), 119.1 (ArC), 112.9 (ArC), 111.1 (ArC), 107.4 (ArC), 64.6 (AkC), 60.6 (AkC), 50.7 (AkC), 21.3 (AkC), 19.9 (AkC), 8.1 (AkC).

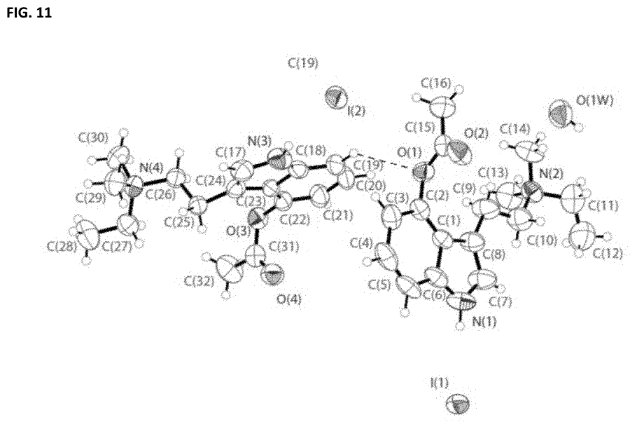

4-AcO-DMET iodide was recrystallized by slow evaporation of an ethanol solution to yield crystals suitable for X-ray diffraction studies. Crystal data, data collection and structure refinement details are summarized in Table 10. FIG. 11 shows the molecular structure of crystalline 4-AcO-DMET iodide hemihydrate showing the atomic labeling. Displacement ellipsoids are drawn at the 50% probability level. There are two distinct tryptammonium cations and two iodides in the asymmetric unit. The solvate water molecule is modeled at 50% occupancy.

TABLE 10
Crystal data

| | |
|---|---|
| 2(I)•2(C₁₆H₂₃N₂O₂)•0.5(H₂O) | F(000) = 818 |
| Mᵣ = 813.53 | Dₓ = 1.471 Mg m⁻³ |
| Monoclinic, P2₁ | Mo Ka radiation, I = 0.71073 Å |
| a = 11.8538 (8) Å | Cell parameters from 9080 reflections |
| b = 10.3179 (7) Å | q = 3.0-25.7° |
| c = 15.0132 (10) Å | m = 1.75 mm⁻¹ |
| β = 90.611 (2)° | T = 297 K |
| V = 1836.1 (2) Å³ | BLOCK, colourless |
| Z = 2 | 0.23 × 0.22 × 0.21 mm |

Data collection

| | |
|---|---|
| Bruker D8 Venture CMOS | 6527 reflections with I > 2s(I) |
| diffractometer | |
| f and w scans | Rᵢₙₜ = 0.029 |
| Absorption correction: multi-scan | qₘₐₓ = 25.7°, qₘᵢₙ = 2.7° |
| SADABS2016/2 (Bruker, 2016/2) was used | |
| for absorption correction. wR2(int) was | |
| 0.0602 before and 0.0524 after correction. | |
| The Ratio of minimum to maximum | |
| transmission is 0.9438. The I/2 correction | |
| factor is Not present. | |
| Tₘᵢₙ = 0.531, Tₘₐₓ = 0.562 | h = −14 ®14 |
| 47080 measured reflections | k = −12 ®12 |
| 6821 independent reflections | l = −18 ®18 |

Refinement

| | |
|---|---|
| Refinement on F² | Hydrogen site location: mixed |
| Least-squares matrix: full | H atoms treated by a mixture of |
| | independent and constrained refinement |
| R[F² > 2s(F²)] = 0.029 | w = 1/[s²(Fₒ²) + (0.0298P)² + 1.2326P] |
| | where P = (Fₒ² + 2F_{c}²)/3 |
| wR(F²) = 0.072 | (D/S)ₘₐₓ = 0.001 |
| S = 1.05 | Dñₘₐₓ = 0.66 e Å⁻³ |
| 6821 reflections | Dñₘᵢₙ = −0.46 e Å⁻³ |
| 410 parameters | Absolute structure: Flack x determined |
| | using 2968 quotients [(I+) − (I−)]/[(I+) + (I−)] |
| | (Parsons, Flack and Wagner, Acta Cryst. |
| | B69 (2013) 249-259). |
| 6 restraints | Absolute structure parameter: −0.009 (5) |

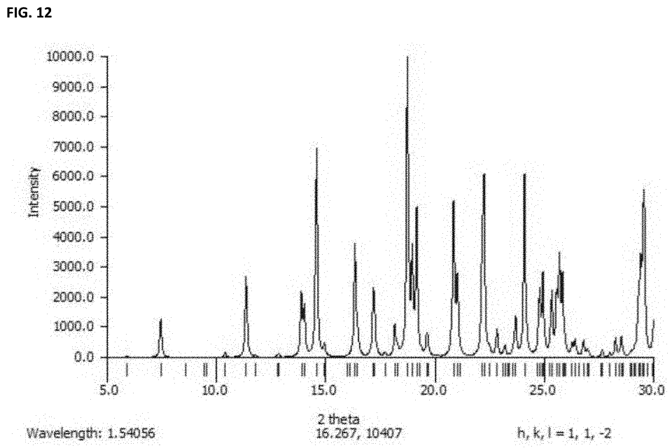

FIG. 12 is a simulated x-ray powder diffraction (XRPD) pattern of crystalline 4-acetoxy-N,N-dimethyl-N-ethyltryptammonium (4-AcO-DMET) iodide hemihydrate from its single crystal data. Crystalline 4-acetoxy-N,N-dimethyl-N-ethyltryptammonium (4-AcO-DMET) iodide hemihydrate may be characterized by the XRPD peaks at 11.4, 14.6 and 19.2°2θ±0.2°2θ as well as by an XRPD pattern substantially similar to FIG. 12.

### Example 7: 4-hydroxy-N,N-dimethyl-N-ethyltryptammonium (4-HO-DMET) Iodide

Synthesis: 150 mg of 4-AcO-DMET iodide was dissolved in 2 mL of DI water, and 10 mL of acetic acid was added. The mixture was refluxed overnight under an atmosphere of nitrogen. The solvent was removed via distillation, yielding an orange sticky oil. The oil was dissolved in a small volume of tetrahydrofuran and acetone. Hexanes was added to the solution, producing a white precipitate. The powder was isolated via vacuum filtration to give 90 mg (67% yield) of pure product. ¹H NMR (400 MHz, D₂O): δ 7.19 (s, 1H, ArH), 7.12-7.07 (m, 2H, ArH), 6.59-6.54 (m, 1H, ArH), 3.60-3.56 (m, 2H, CH₂), 3.46 (q, J=7.3 Hz, 2H, CH₂), 3.35-3.31 (m, 2H, CH₂), 3.12 (s, 6H, CH₃), 1.39 (t, J=7.3 Hz, 3H, CH₃); ¹³C NMR (100 MHz, D₂O): δ 150.6 (ArC), 139.3 (ArC), 123.9 (ArC), 116.6 (ArC), 108.8 (ArC), 105.2 (ArC), 104.4 (ArC), 65.0 (AkC), 60.3 (AkC), 50.7 (AkC), 20.7 (AkC), 8.0 (AkC).

### Example 8: 4-acetoxy-N,N-dimethyl-N-n-propyltryptammonium (4-AcO-DMPT) Iodide

Synthesis: 323 mg of 4-AcO-DMT fumarate was dissolved in 30 mL of tetrahydrofuran, and 6 mL of 1-iodopropane was added. The mixture was refluxed overnight under an atmosphere of nitrogen. This resulted in the precipitation of a white powder from the yellow solution. The precipitate was isolated via vacuum filtration to give a white powder, which was washed with diethyl ether to yield 314 mg of pure product (85% yield). ¹H NMR (400 MHz, D₂O): δ 7.46 (dd, J=8.2, 0.5 Hz, 1H, ArH), 7.33 (s, 1H, ArH), 7.26 (t, J=7.9 Hz, 1H, ArH), 6.90 (d, J=7.7 Hz, 1H, ArH), 3.61-3.57 (m, 2H, CH₂), 3.35-3.29 (m, 2H, CH₂), 3.27-3.21 (m, 2H, CH₂), 3.14 (s, 6H, CH₃), 2.47 (s, 3H, (CO)CH₃), 1.81-1.72 (m, 2H, CH₂), 0.91 (t, J=7.3 Hz, 3H, CH₃); ¹³C NMR (100 MHz, D₂O): δ 174.3 (CO), 143.6 (ArC), 139.3 (ArC), 125.8 (ArC), 123.0 (ArC), 119.1 (ArC), 112.9 (ArC), 111.1 (ArC), 107.5 (ArC), 66.4 (AkC), 65.1 (AkC), 51.3 (AkC), 21.3 (AkC), 20.1 (AkC), 16.3 (AkC), 10.4 (AkC).

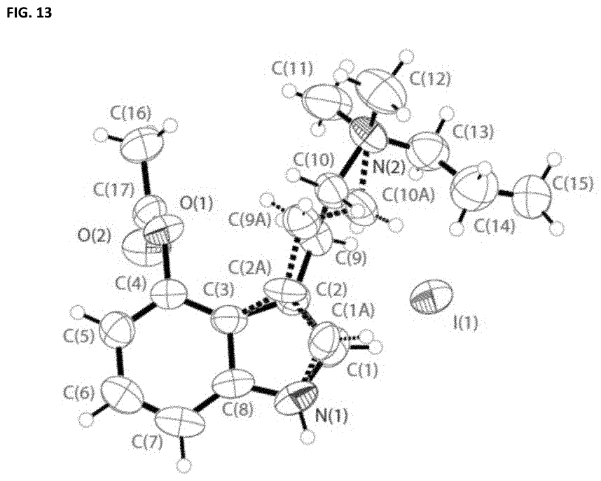

4-AcO-DMPT iodide was recrystallized by slow evaporation of an ethanol solution to yield crystals suitable for X-ray diffraction studies. Crystal data, data collection and structure refinement details are summarized in Table 11. FIG. 13 shows the molecular structure of crystalline 4-AcO-DMPT iodide showing the atomic labeling. Displacement ellipsoids are drawn at the 50% probability level. Dashed bonds indicate a disordered component in the structure.

TABLE 11
Crystal data

| | |
|---|---|
| I•C₁₇H₂₅N₂O₂ | F(000) = 420 |
| Mᵣ = 416.29 | Dₓ = 1.495 Mg m⁻³ |
| Monoclinic, P2₁ | Mo Ka radiation, I = 0.71073 Å |
| a = 7.7067 (4) Å | Cell parameters from 9480 reflections |
| b = 10.3424 (4) Å | q = 2.7-25.7° |
| c = 11.6302 (6) Å | m = 1.74 mm⁻¹ |
| β = 94.222 (2)° | T = 273 K |
| V = 924.48 (8) Å³ | BLOCK, colourless |
| Z = 2 | 0.23 × 0.2 × 0.07 mm |

Data collection

| | |
|---|---|
| Bruker D8 Venture CMOS | 3251 reflections with I > 2s(I) |
| diffractometer | |
| f and w scans | Rᵢₙₜ = 0.030 |
| Absorption correction: multi-scan | qₘₐₓ = 25.8°, qₘᵢₙ = 2.7° |
| SADABS2016/2 (Bruker, 2016/2) was used | |
| for absorption correction. wR2(int) was | |
| 0.0659 before and 0.0527 after correction. | |
| The Ratio of minimum to maximum | |
| transmission is 0.8900. The I/2 correction | |
| factor is Not present. | |
| Tₘᵢₙ = 0.663, Tₘₐₓ = 0.745 | h = −9 ®9 |
| 32264 measured reflections | k = −12 ®12 |
| 3428 independent reflections | l = −14 ®14 |

Refinement

| | |
|---|---|
| Refinement on F² | Hydrogen site location: mixed |
| Least-squares matrix: full | H atoms treated by a mixture of |
| | independent and constrained refinement |
| R[F² > 2s(F²)] = 0.035 | w = 1/[s²(Fₒ²) + (0.0233P)² + 1.3282P] |
| | where P = (Fₒ² + 2F_{c}²)/3 |
| wR(F²) = 0.081 | (D/s)ₘₐₓ < 0.001 |
| S = 1.04 | Dñₘₐₓ = 1.09 e Å⁻³ |
| 3428 reflections | Dñₘᵢₙ = −0.78 e Å⁻³ |
| 243 parameters | Absolute structure: Flack x determined |
| | using 1473 quotients [(I+) − (I−)]/[(I+) + (I−)] |
| | (Parsons, Flack and Wagner, Acta Cryst. |
| | B69 (2013) 249-259). |
| 35 restraints | Absolute structure parameter: 0.009 (7) |

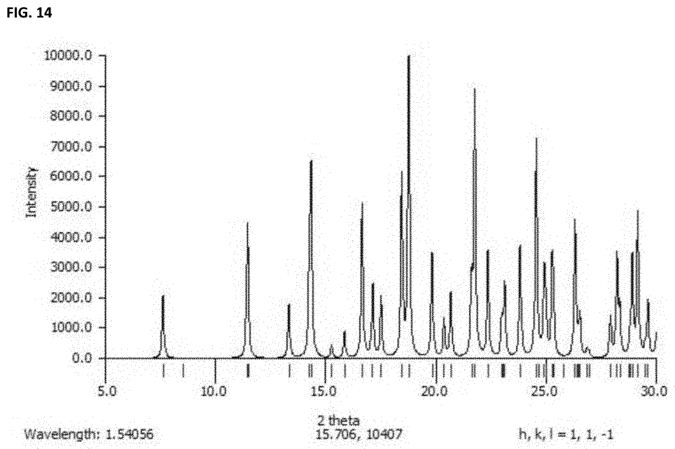

FIG. 14 is a simulated x-ray powder diffraction (XRPD) pattern of crystalline 4-acetoxy-N,N-dimethyl-N-n-propyltryptammonium (4-AcO-DMPT) iodide from its single crystal data. Crystalline 4-acetoxy-N,N-dimethyl-N-n-propyltryptammonium (4-AcO-DMPT) iodide may be characterized by the XRPD peaks at 11.5, 16.7 and 19.8°2θ±0.2°2θ as well as by an XRPD pattern substantially similar to FIG. 14.

### Example 9: 4-hydroxy-N,N-dimethyl-N-n-propyltryptammonium (4-HO-DMPT) Iodide

Synthesis: 200 mg of 4-AcO-DMPT iodide was dissolved in 3 mL of deionized (DI) water, and 10 mL of acetic acid was added. The mixture was refluxed overnight under an atmosphere of nitrogen. The solvent was removed via distillation, yielding an orange sticky oil. The oil was dissolved in a small volume of tetrahydrofuran and acetone. Hexanes was then added to the solution, producing a light green precipitate. This powder was isolated via vacuum filtration and washed with diethyl ether to give 157 mg (87% yield) of pure product. ¹H NMR (400 MHz, D₂O): δ 7.17 (s, 1H, ArH), 7.12-7.07 (m, 2H, ArH), 6.57-6.55 (m, 1H, ArH), 3.57-3.53 (m, 2H, CH₂), 3.32-3.26 (m, 4H, CH₂), 3.11 (s, 6H, CH₃), 1.85-1.75 (m, 2H, CH₂), 0.95 (t, J=7.3 Hz, 3H, CH₃); ¹³C NMR (100 MHz, D₂O): δ 150.6 (ArC), 139.3 (ArC), 123.9 (ArC), 116.7 (ArC), 108.9 (ArC), 105.2 (ArC), 104.4 (ArC), 66.1 (AkC), 65.4 (AkC), 51.3 (AkC), 20.8 (AkC), 16.2 (AkC), 10.4 (AkC).

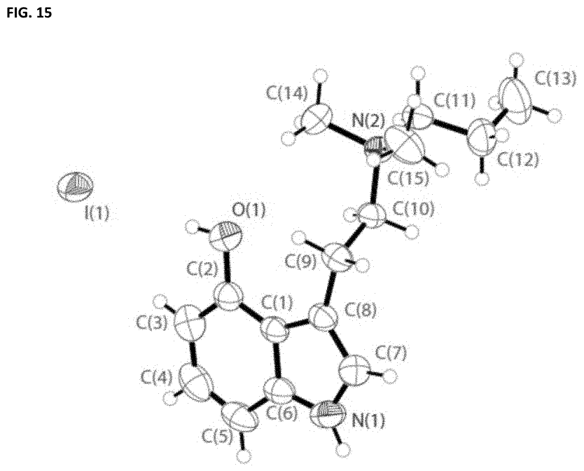

4-HO-DMPT iodide was recrystallized by slow evaporation of an ethanol solution to yield crystals suitable for X-ray diffraction studies. Crystal data, data collection and structure refinement details are summarized in Table 12. FIG. 15 shows the molecular structure of 4-HO-DMPT iodide showing the atomic labeling. Displacement ellipsoids are drawn at the 50% probability level.

TABLE 12
Crystal data

| | |
|---|---|
| I•C₁₅H₂₃N₂O | F(000) = 752 |
| Mᵣ = 374.25 | Dₓ = 1.487 Mg m⁻³ |
| Monoclinic, P2₁/c | Mo Ka radiation, I = 0.71073 Å |
| a = 9.4296 (8) Å | Cell parameters from 9954 reflections |
| b = 14.1816 (11) Å | q = 2.7-25.7° |
| c = 13.2586 (10) Å | m = 1.91 mm⁻¹ |
| β = 109.423 (3)° | T = 297 K |
| V = 1672.1 (2) Å³ | BLOCK, colourless |
| Z = 4 | 0.25 × 0.2 × 0.19 mm |

Data collection

| | |
|---|---|
| Bruker D8 Venture CMOS | 2808 reflections with I > 2s(I) |
| diffractometer | |
| f and w scans | Rᵢₙₜ = 0.025 |
| Absorption correction: multi-scan | qₘₐₓ = 25.7°, qₘᵢₙ = 2.7° |
| SADABS2016/2 (Bruker, 2016/2) was used | |
| for absorption correction. wR2(int) was | |
| 0.0578 before and 0.0440 after correction. | |
| The Ratio of minimum to maximum | |
| transmission is 0.9109. The I/2 correction | |
| factor is Not present. | |
| Tₘᵢₙ = 0.679, Tₘₐₓ = 0.745 | h = −11 ®11 |
| 38248 measured reflections | k = −17 ®17 |
| 3102 independent reflections | l = −16 ®16 |

Refinement

| | |
|---|---|
| Refinement on F² | Hydrogen site location: mixed |
| Least-squares matrix: full | H atoms treated by a mixture of |
| | independent and constrained refinement |
| R[F² > 2s(F²)] = 0.027 | w = 1/[s²(Fₒ²) + (0.0259P)² + 1.4322P] |
| | where P = (Fₒ² + 2F_{c}²)/3 |
| wR(F²) = 0.069 | (D/S)ₘₐₓ = 0.001 |
| S = 1.05 | Dñₘₐₓ = 0.85 e Å⁻³ |
| 3102 reflections | Dñₘᵢₙ = −0.64 e Å⁻³ |
| 182 parameters | Extinction correction: SHELXL2018/3 |
| | (Sheldrick 2018), |
| | Fc* = kFc[1 + 0.001 × Fc²I³/sin(2q)]^{-1/4} |
| 16 restraints | Extinction coefficient: 0.0082 (16) |
| Primary atom site location: structure- | |
| invariant direct methods | |

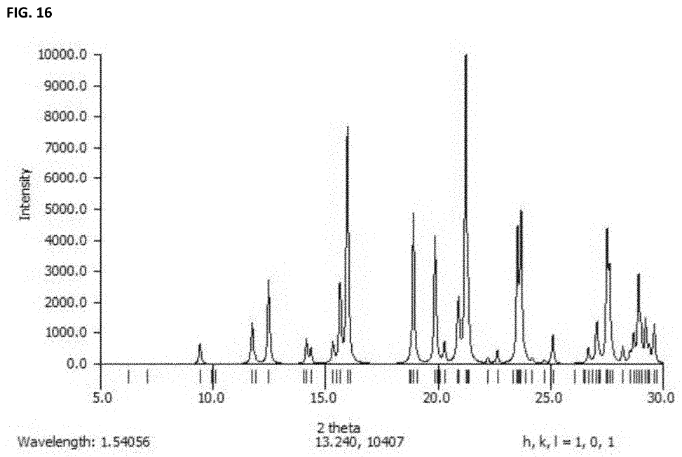

FIG. 16 is a simulated x-ray powder diffraction (XRPD) pattern of crystalline 4-hydroxy-N,N-dimethyl-N-n-propyltryptammonium (4-HO-DMPT) iodide from its single crystal data. Crystalline 4-hydroxy-N,N-dimethyl-N-n-propyltryptammonium (4-HO-DMPT) iodide may be characterized by the XRPD peaks at 12.5, 18.9 and 19.9° 2θ±0.2°2θ as well as by an XRPD pattern substantially similar to FIG. 16.

### Example 10: 4-acetoxy-N,N-dimethyl-N-isopropyltryptammonium (4-AcO-DMiPT) Iodide

Synthesis: 320 mg of 4-AcO-DMT fumarate was dissolved in 30 mL of tetrahydrofuran, and 12 mL of 2-iodopropane was added. The mixture was refluxed overnight under an atmosphere of nitrogen. A mixture of orange/yellow solid and yellow liquid was obtained. The liquid was decanted and the remaining solid was triturated with ethyl acetate to yield a white powder. The powder was isolated via vacuum filtration to give 151 mg of pure product (41% yield). ¹H NMR (400 MHz, D₂O): δ 7.46 (dd, J=8.2, 0.7 Hz, 1H, ArH), 7.33 (s, 1H, ArH), 7.26 (t, J=7.9 Hz, 1H, ArH), 6.89 (dd, J=7.7, 0.6 Hz, 1H, ArH), 3.86-3.76 (sep, J=6.6 Hz, 1H, CH), 3.57-3.53 (m, 2H, CH₂), 3.26-3.22 (m, 2H, CH₂), 3.07 (s, 6H, CH₃), 2.47 (s, 3H, (CO)CH₃), 1.41 (d, J=6.6 Hz, 6H, CH₃); ¹³C NMR (100 MHz, D₂O): δ 174.2 (CO), 143.6 (ArC), 139.2 (ArC), 125.7 (ArC), 123.0 (ArC), 119.1 (ArC), 113.0 (ArC), 111.1 (ArC), 107.5 (ArC), 66.0 (AkC), 63.5 (AkC), 48.1 (AkC), 21.3 (AkC), 19.7 (AkC), 16.2 (AkC).

### REFERENCES

Bradley, R. J. & Johnston, V. S. (1970). Origin and Mechanism of Hallucinations, edited by W. Keup, pp. 333-344. New York: Plenum Press.
Cameron, L. P. & Olson, D. E. (2018). ACS Chem. Neurosci. 9, 2344-2357.
Carhart-Harris, R. L. & Goodwin, G. M. (2017). Neuropsychopharmacology, 42, 2105-2113.
Dinis-Oliveira, R. J. (2017). Drug Metab. Rev. 49, 84-91.
Johnson, M. W. & Griffiths, R. R. (2017). Neurotherapeutics 14, 734-740.
McKenna, D. J., Repke, D. B., Lo, L. & Peroutka, S. J. (1990). Neuropharmacology, 29, 193-198.
Nichols, D. E. (2012). WIREs Membr. Transp. Signal. 1, 559-579.
Repke, D. B., Grotjahn, D. B. & Shulgin, A. T. (1985). J. Med. Chem. 28, 892-896.
C. Lenz, J. Wick and D. Hoffmeister, J. Nat. Prod., 2017, 80, 2835-2838.
A. M. Sherwood, A. L. Halberstadt, A. K. Klein, J. D. McCorvy, K. W. Kaylo, R. B. Kargbo and P. Meisenheimer, J. Nat. Prod., Article ASAP, DOI: 10.1021/acs.jnatprod.9b01061.
N. Jensen, J. Gartz and H. Laatsch, Planta Med., 2006, 72, 665-666.
J. Gartz, Int. J. Crude Drug Res., 1989, 27, 141-144.
B. L. Roth, W. K. Kroeze, S. Patel and E. Lopez, The Neuroscientist, 2000, 6, 252-262.

1-53. (canceled)

54. A compound of formula (I): 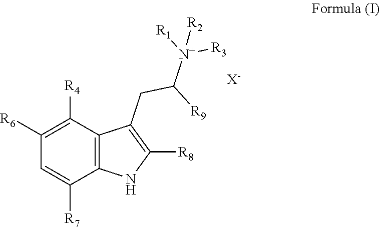
wherein
R₁, R₂ and R₃ are each independently a straight chain or branched C₁-C₆ alkyl or C₂-C₆ alkenyl;
R₄ is hydrogen, hydroxyl, C₁-C₆ alkoxy, —OC(O)R₅ or —OC(O)OR₅;
R₅ is a straight chain or branched C₁-C₆ alkyl;
R₆ is hydrogen, hydroxyl, C₁-C₆ alkoxy, —OC(O)R₅, —OC(O)OR₅, or a straight chain or branched C₁-C₆ alkyl;
R₇, R₈ and R₉ are each independently hydrogen or a straight chain or branched C₁-C₆ alkyl; and
X⁻ is a pharmaceutically acceptable anion.

55. The compound of claim 54, with the proviso that when R₄, R₇, R₈ and R₉ are hydrogen and R₆ is hydroxy or methoxy, R₁, R₂, and R₃ are not each methyl.

56. The compound of claim 54, with the proviso that when R₄, R₇, R₈ and R₉ are hydrogen, R₆ is hydroxy, and R₁ and R₂ are methyl, R₃ is not ethyl.

57. The compound of claim 54, wherein at least one of R₄, R₆, R₇, R₈, and R₉ is not hydrogen.

58. The compound of claim 54, with the proviso that R₁, R₂ and R₃ are not all methyl when R₄ or Reis hydroxyl and R₇, R₈, and R₉ are hydrogen.

59. A pharmaceutical composition comprising a therapeutically effective amount of a compound of claim 54 and a pharmaceutically acceptable excipient.

60. A composition comprising as a first active component: a compound according to claim 54; and a second active component selected from at least one of (a) a serotonergic drug, (b) a purified psilocybin derivative, (c) a purified cannabinoid, or (d) a purified terpene; and a pharmaceutically acceptable excipient.

61. A method of preventing or treating a psychological disorder comprising:
identifying a subject in need of treatment or prevention; and
administering to a subject in need thereof a therapeutically effective amount of a compound according to claim 54.

62. A method of generating a dialkyltryptamine compound in situ in a patient, comprising contacting a compound according to claim 54 with an enzyme in the patient capable of nitrogen dealkylation.

63. A tryptamine compound of formula (II): 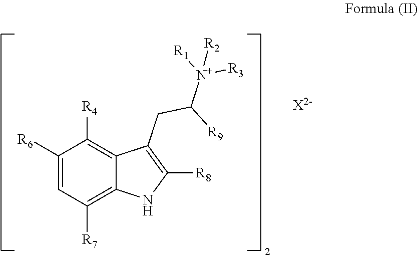
wherein
R₁, R₂ and R₃ are independently selected from straight chain or branched C₁-C₆ alkyl or a straight chain or branched C₂-C₆ alkenyl;
R₄ and R₆ are independently chosen from hydrogen, hydroxyl, —OR₅, —OC(O)R₅, and —OC(O)OR₅;
R₅ is a straight chain or branched C₁-C₆ alkyl or a substituted or unsubstituted aryl;
R₇, R₈ and R₉ are each independently hydrogen or a straight chain or branched C₁-C₆ alkyl; and
and X²⁻ pharmaceutically-acceptable dianion.

64. A pharmaceutical composition comprising a therapeutically effective amount of a compound of claim 63 and a pharmaceutically acceptable excipient.

65. A composition comprising as a first active component: a compound according to claim 63;
and a second active component selected from at least one of (a) a serotonergic drug, (b) a purified psilocybin derivative, (c) a purified cannabinoid, or (d) a purified terpene; and a pharmaceutically acceptable excipient.

66. A method of preventing or treating a psychological disorder comprising:
identifying a subject in need of treatment or prevention; and
administering to a subject in need thereof a therapeutically effective amount of a compound according to claim 63.

67. A method of generating a dialkyltryptamine compound in situ in a patient, comprising contacting a compound according to claim 63 with an enzyme in the patient capable of nitrogen dealkylation.

68. A zwitterionic tryptamine compound of formula (III) 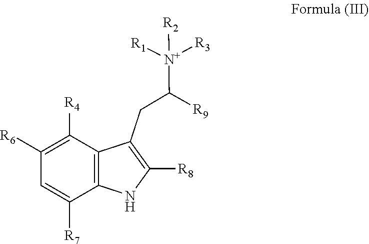
wherein
R₁, R₂ and R₃ are each independently a straight chain or branched C₁-C₆ alkyl or C₂-C₆ alkenyl;
R₄ is hydrogen, —O⁻, C₁-C₆ alkoxy, —OC(O)R₅, —OC(O)O⁻, —OSO₂O⁻, or —OP(O)OHO⁻;
R₅ is a straight chain or branched C₁-C₆ alkyl;
R₆ is hydrogen, —O⁻, C₁-C₆ alkoxy, —OC(O)R₅, —OC(O)OR₅, or a straight chain or branched C₁-C₆ alkyl; and
R₇, R₈ and R₉ are each independently hydrogen or a straight chain or branched C₁-C₆ alkyl, wherein at least one of R₄ or R₆ is —O⁻, —OC(O)O⁻, —OSO₂O⁻, or —OP(O)OHO⁻, provided that R₄ is not —OP(O)OHO⁻ when R₁, R₂ and R₃ are each methyl.

69. The zwitterionic compound of claim 68, wherein R₆ is not —O⁻ when R₁, R₂ and R₃ are methyl, R₄ and R₇ are hydrogen, R₈ is methyl, and R₉ is hydrogen.

70. A pharmaceutical composition comprising a therapeutically effective amount of a compound of claim 68 and a pharmaceutically acceptable excipient.

71. A composition comprising as a first active component: a compound according to claim 68;
and a second active component selected from at least one of (a) a serotonergic drug, (b) a purified psilocybin derivative, (c) a purified cannabinoid, or (d) a purified terpene; and a pharmaceutically acceptable excipient.

72. A method of preventing or treating a psychological disorder comprising:
identifying a subject in need of treatment or prevention; and
administering to a subject in need thereof a therapeutically effective amount of a compound according to claim 68.

73. A method of generating a dialkyltryptamine compound in situ in a patient, comprising contacting a compound according to claim 68 with an enzyme in the patient capable of nitrogen dealkylation.
